SEQUENCE LISTING (208 sequences)

SEQ ID NO: 1 - DNA, 780 bp, Homo sapiens
caggtccagctggtgcagtctggggctgaggtgaagaagcctgggtcctcggtgagggtctcctgcaaggcttctggaggctccttcagcagctatggtatccactgggtgcaacaggcccctggtcaagggcttgagtggatggggaggatcatccctgtccttcgtataagaaactacgcacagaagttccacgacagagtcaccatagacgcggacacatccacgggcacagcctacatggaattgagcagcctgacatctgacgacacggccgtgtattactgtgcgggttctcggcaaggcgttgccccgactggctactggggccaggggacaatggtcaccgtctcgagtggaggcggcggttcaggcggaggtggctctggcggtggcggaagtgcacaggctgtgctgactcagccgtcttccctctctgcatctcctggagcatcagccagtctcacctgcaacttacgcagtggcatcgatgttggtacctacaggatatactggtaccagcagaagccagggagtcctccccagtatctcctgaggtacaaatcagactcagataagcagcagggctctggagtccccagccgcttctctggaaccaaggatgcttcggccaatgcagggattttactcatctctgggctccagtctgaggatgaggctgactattactgtatgatttggcacagcaacggttgggtgttcggcggagggaccaagctgaccgtcctaggtgcggccgcacatcatcatcaccatcat

SEQ ID NO: 2 - protein, 260 aa, Homo sapiens
QVQLVQSGAEVKKPGSSVRVSCKASGGSFSSYGIHWVQQAPGQGLEWMGRIIPVLRIRNYAQKFHDRVTIDADTSTGTAYMELSSLTSDDTAVYYCAGSRQGVAPTGYWGQGTMVTVSSGGGGSGGGGSGGGGSAQAVLTQPSSLSASPGASASLTCNLRSGIDVGTYRIYWYQQKPGSPPQYLLRYKSDSDKQQGSGVPSRFSGTKDASANAGILLISGLQSEDEADYYCMIWHSNGWVFGGGTKLTVLGAAAHHHHHH

SEQ ID NO: 3 - DNA, 357 bp, Homo sapiens
caggtccagctggtgcagtctggggctgaggtgaagaagcctgggtcctcggtgagggtctcctgcaaggcttctggaggctccttcagcagctatggtatccactgggtgcaacaggcccctggtcaagggcttgagtggatggggaggatcatccctgtccttcgtataagaaactacgcacagaagttccacgacagagtcaccatagacgcggacacatccacgggcacagcctacatggaattgagcagcctgacatctgacgacacggccgtgtattactgtgcgggttctcggcaaggcgttgccccgactggctactggggccaggggacaatggtcaccgtctcgagt

SEQ ID NO: 4 - protein, 119 aa, Homo sapiens
QVQLVQSGAEVKKPGSSVRVSCKASGGSFSSYGIHWVQQAPGQGLEWMGRIIPVLRIRNYAQKFHDRVTIDADTSTGTAYMELSSLTSDDTAVYYCAGSRQGVAPTGYWGQGTMVTVSS

SEQ ID NO: 5 - DNA, 345 bp, Homo sapiens
caggctgtgctgactcagccgtcttccctctctgcatctcctggagcatcagccagtctcacctgcaacttacgcagtggcatcgatgttggtacctacaggatatactggtaccagcagaagccagggagtcctccccagtatctcctgaggtacaaatcagactcagataagcagcagggctctggagtccccagccgcttctctggaaccaaggatgcttcggccaatgcagggattttactcatctctgggctccagtctgaggatgaggctgactattactgtatgatttggcacagcaacggttgggtgttcggcggagggaccaagctgaccgtccta

SEQ ID NO: 6 - protein, 115 aa, Homo sapiens
QAVLTQPSSLSASPGASASLTCNLRSGIDVGTYRIYWYQQKPGSPPQYLLRYKSDSDKQQGSGVPSRFSGTKDASANAGILLISGLQSEDEADYYCMIWHSNGWVFGGGTKLTVL

SEQ ID NO: 7 - DNA, 765 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaacaggacggaaactggggccgatactttgactactggggccgggggacaatggtcaccgtctcgagtggaggcggcggttcaggcggaggtggctctggcggtggcggaagtgcactttcttctgagctgactcaggaccctgatgtgtctgtggccttgggacagacagtcaggatcacatgccaaggagacagcctcagaagctattatgcaagctggtaccagcagaaggcaggacaggcccctgtagttgtcgtctttggtaaaaacaagcggccctcagggctcccagaccgcttctctggctccagctcaggaaacacagcttccttgaccatcactggggctcaggcggaagatgaggctgactattactgtagctcccgggacagcagtggtaaacgccgggtcttcggaactgggaccaaggtcaccgtcctaggtgcggccgcacatcatcatcaccatcat

SEQ ID NO: 8 - protein, 255 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKQDGNWGRYFDYWGRGTMVTVSSGGGGSGGGGSGGGGSALSSELTQDPDVSVALGQTVRITCQGDSLRSYYASWYQQKAGQAPVVVVFGKNKRPSGLPDRFSGSSSGNTASLTITGAQAEDEADYYCSSRDSSGKRRVFGTGTKVTVLGAAAHHHHHH

SEQ ID NO: 9 - DNA, 360 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaacaggacggaaactggggccgatactttgactactggggccgggggacaatggtcaccgtctcgagt

SEQ ID NO: 10 - protein, 120 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKQDGNWGRYFDYWGRGTMVTVSS

SEQ ID NO: 11 - DNA, 279 bp, Homo sapiens
cagacagtcaggatcacatgccaaggagacagcctcagaagctattatgcaagctggtaccagcagaaggcaggacaggcccctgtagttgtcgtctttggtaaaaacaagcggccctcagggctcccagaccgcttctctggctccagctcaggaaacacagcttccttgaccatcactggggctcaggcggaagatgaggctgactattactgtagctcccgggacagcagtggtaaacgccgggtcttcggaactgggaccaaggtcaccgtccta

SEQ ID NO: 12 - protein, 93 aa, Homo sapiens
QTVRITCQGDSLRSYYASWYQQKAGQAPVVVVFGKNKRPSGLPDRFSGSSSGNTASLTITGAQAEDEADYYCSSRDSSGKRRVFGTGTKVTVL

SEQ ID NO: 13 - DNA, 753 bp, Homo sapiens
gaggtgcagctgttggagtctgggggaggcttggtacggcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagataagcgtcttacatattggggccaagggacaatggtcaccgtctcgagtggaggcggcggttcaggcggaggtggctctggcggtggcggaagtgcacagtctgtcgtgacgcagccgccctcagtgtccgcggccccaggacagaaggtcaccatctcctgctctggaagcagctccaacattgggaataattatgtatcatggtaccagcagctcccaggaacagcccccaaactcctcatttatgacaataataagcgaccctcagggattcctgaccgattctctggctccaagtctggcacgtccgccaccctgggcatcaccggactccagactggggacgaggccgattattactgcggaacatgggatagcagcctgagtgctcgggtgttcggcggagggaccaaggtcaccgtcctaggtgcggccgcacatcatcatcaccatcac

SEQ ID NO: 14 - protein, 251 aa, Homo sapiens
EVQLLESGGGLVRPGGSLRLSCAASGFTFSSYAMSWVRQAPGKGLEWVSAISGSGGSTYYADSVKGRFTISRDNSKNTLYLQMNSLRAEDTAVYYCARDKRLTYWGQGTMVTVSSGGGGSGGGGSGGGGSAQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSARVFGGGTKVTVLGAAAHHHHHH

SEQ ID NO: 15 - DNA, 345 bp, Homo sapiens
gaggtgcagctgttggagtctgggggaggcttggtacggcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagataagcgtcttacatattggggccaagggacaatggtcaccgtctcgagt

SEQ ID NO: 16 - protein, 115 aa, Homo sapiens
EVQLLESGGGLVRPGGSLRLSCAASGFTFSSYAMSWVRQAPGKGLEWVSAISGSGGSTYYADSVKGRFTISRDNSKNTLYLQMNSLRAEDTAVYYCARDKRLTYWGQGTMVTVSS

SEQ ID NO: 17 - DNA, 330 bp, Homo sapiens
cagtctgtcgtgacgcagccgccctcagtgtccgcggccccaggacagaaggtcaccatctcctgctctggaagcagctccaacattgggaataattatgtatcatggtaccagcagctcccaggaacagcccccaaactcctcatttatgacaataataagcgaccctcagggattcctgaccgattctctggctccaagtctggcacgtccgccaccctgggcatcaccggactccagactggggacgaggccgattattactgcggaacatgggatagcagcctgagtgctcgggtgttcggcggagggaccaaggtcaccgtccta

SEQ ID NO: 18 - protein, 110 aa, Homo sapiens
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSARVFGGGTKVTVL

SEQ ID NO: 19 - protein, 5 aa, Homo sapiens
SYGIH

SEQ ID NO: 20 - protein, 17 aa, Homo sapiens
RIIPVLRIRNYAQKFHD

SEQ ID NO: 21 - protein, 10 aa, Homo sapiens
SRQGVAPTGY

SEQ ID NO: 22 - protein, 14 aa, Homo sapiens
NLRSGIDVGTYRIY

SEQ ID NO: 23 - protein, 10 aa, Homo sapiens
KSDSDKQQGS

SEQ ID NO: 24 - protein, 9 aa, Homo sapiens
MIWHSNGWV

SEQ ID NO: 25 - protein, 5 aa, Homo sapiens
SYAMS

SEQ ID NO: 26 - protein, 17 aa, Homo sapiens
AISGSGGSTYYADSVKG

SEQ ID NO: 27 - protein, 4 aa, Homo sapiens
QDGN

SEQ ID NO: 28 - protein, 11 aa, Homo sapiens
QGDSLRSYYAS

SEQ ID NO: 29 - protein, 7 aa, Homo sapiens
GKNKRPS

SEQ ID NO: 30 - protein, 11 aa, Homo sapiens
SSRDSSGKRRV

SEQ ID NO: 31 - protein, 5 aa, Homo sapiens
SYAMS

SEQ ID NO: 32 - protein, 17 aa, Homo sapiens
AISGSGGSTYYADSVKG

SEQ ID NO: 33 - protein, 6 aa, Homo sapiens
DKRLTY

SEQ ID NO: 34 - protein, 13 aa, Homo sapiens
SGSSSNIGNNYVS

SEQ ID NO: 35 - protein, 7 aa, Homo sapiens
DNNKRPS

SEQ ID NO: 36 - protein, 11 aa, Homo sapiens
GTWDSSLSARV

SEQ ID NO: 37 - protein, 468 aa, Homo sapiens
MGWSCIILFLVATATGAHSQVQLVQSGAEVKKPGSSVRVSCKASGGSFSSYGIHWVRQAPGQGLEWMGRIIPVLRIRNYAQKFHDRVTITADTSTSTAYMELSSLTSDDTAVYYCAGSRQGVAPTGYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 108 aa, Homo sapiens
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 28 aa, Homo sapiens
  MOD_RES (5): Sulfated Tyr
  MOD_RES (7): Sulfated Tyr
  MOD_RES (10): Sulfated Tyr
QATEYEYLDYDFLPETEPPRPMMDDDDK

SEQ ID NO: 43 - protein, 9 aa, Homo sapiens
  MOD_RES (2): Sulfated Tyr
  MOD_RES (4): Sulfated Tyr
  MOD_RES (7): Sulfated Tyr
EYEYLDYDF

SEQ ID NO: 44 - protein, 9 aa, Artificial Sequence
  Description of Artificial Sequence Synthetic peptide
  MOD_RES (2): Sulfated Tyr
EYEYLDYDF

SEQ ID NO: 45 - protein, 9 aa, Artificial Sequence
  Description of Artificial Sequence Synthetic peptide
  MOD_RES (7): Sulfated Tyr
EYEYLDYDF

SEQ ID NO: 46 - protein, 19 aa, Homo sapiens
QATEYEYLDYDFLPETEPP

SEQ ID NO: 47 - protein, 28 aa, Homo sapiens
QATEYEYLDYDFLPETEPPRPMMDDDDK

SEQ ID NO: 48 - protein, 28 aa, Homo sapiens
  MOD_RES (5): Sulfated Tyr
QATEYEYLDYDFLPETEPPRPMMDDDDK

SEQ ID NO: 49 - protein, 28 aa, Homo sapiens
  MOD_RES (7): Sulfated Tyr
QATEYEYLDYDFLPETEPPRPMMDDDDK

SEQ ID NO: 50 - protein, 28 aa, Homo sapiens
  MOD_RES (10): Sulfated Tyr
QATEYEYLDYDFLPETEPPRPMMDDDDK

SEQ ID NO: 51 - protein, 28 aa, Homo sapiens
  MOD_RES (7): Sulfated Tyr
  MOD_RES (10): Sulfated Tyr
QATEYEYLDYDFLPETEPPRPMMDDDDK

SEQ ID NO: 52 - protein, 28 aa, Homo sapiens
  MOD_RES (5): Sulfated Tyr
  MOD_RES (10): Sulfated Tyr
QATEYEYLDYDFLPETEPPRPMMDDDDK

SEQ ID NO: 53 - protein, 28 aa, Homo sapiens
  MOD_RES (5): Sulfated Tyr
  MOD_RES (7): Sulfated Tyr
QATEYEYLDYDFLPETEPPRPMMDDDDK

SEQ ID NO: 54 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 55 - protein, 13 aa, Artificial Sequence
  Description of Artificial Sequence Synthetic peptide
QATEYEYLDYDFL

SEQ ID NO: 56 - protein, 13 aa, Artificial Sequence
  Description of Artificial Sequence Synthetic peptide
  MOD_RES (5): Sulfated Tyr
QATEYEYLDYDFL

SEQ ID NO: 57 - protein, 13 aa, Artificial Sequence
  Description of Artificial Sequence Synthetic peptide
  MOD_RES (7): Sulfated Tyr
QATEYEYLDYDFL

SEQ ID NO: 58 - protein, 13 aa, Artificial Sequence
  Description of Artificial Sequence Synthetic peptide
  MOD_RES (10): Sulfated Tyr
QATEYEYLDYDFL

SEQ ID NO: 59 - protein, 13 aa, Artificial Sequence
  Description of Artificial Sequence Synthetic peptide
  MOD_RES (5): Sulfated Tyr
  MOD_RES (7): Sulfated Tyr
QATEYEYLDYDFL

SEQ ID NO: 60 - protein, 13 aa, Artificial Sequence
  Description of Artificial Sequence Synthetic peptide
  MOD_RES (5): Sulfated Tyr
  MOD_RES (10): Sulfated Tyr
QATEYEYLDYDFL

SEQ ID NO: 61 - protein, 13 aa, Artificial Sequence
  Description of Artificial Sequence Synthetic peptide
  MOD_RES (7): Sulfated Tyr
  MOD_RES (10): Sulfated Tyr
QATEYEYLDYDFL

SEQ ID NO: 62 - protein, 12 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDF

SEQ ID NO: 63 - protein, 11 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYD

SEQ ID NO: 64 - protein, 12 aa, Artificial Sequence
  Description of Artificial Sequence Synthetic peptide
  MOD_RES (4): Sulfated Tyr
  MOD_RES (6): Sulfated Tyr
  MOD_RES (9): Sulfated Tyr
ATEYEYLDYDFL

SEQ ID NO: 65 - protein, 11 aa, Artificial Sequence
  Description of Artificial Sequence Synthetic peptide
  MOD_RES (4): Sulfated Tyr
  MOD_RES (6): Sulfated Tyr
  MOD_RES (9): Sulfated Tyr
ATEYEYLDYDF

SEQ ID NO: 66 - protein, 10 aa, Artificial Sequence
  Description of Artificial Sequence Synthetic peptide
  MOD_RES (4): Sulfated Tyr
  MOD_RES (6): Sulfated Tyr
  MOD_RES (9): Sulfated Tyr
ATEYEYLDYD

SEQ ID NO: 67 - protein, 11 aa, Artificial Sequence
  Description of Artificial Sequence Synthetic peptide
  MOD_RES (3): Sulfated Tyr
  MOD_RES (5): Sulfated Tyr
  MOD_RES (8): Sulfated Tyr
TEYEYLDYDFL

SEQ ID NO: 68 - protein, 10 aa, Artificial Sequence
  Description of Artificial Sequence Synthetic peptide
  MOD_RES (3): Sulfated Tyr
  MOD_RES (5): Sulfated Tyr
  MOD_RES (8): Sulfated Tyr
TEYEYLDYDF

SEQ ID NO: 69 - protein, 9 aa, Artificial Sequence
  Description of Artificial Sequence Synthetic peptide
  MOD_RES (3): Sulfated Tyr
  MOD_RES (5): Sulfated Tyr
  MOD_RES (8): Sulfated Tyr
TEYEYLDYD

SEQ ID NO: 70 - protein, 10 aa, Artificial Sequence
  Description of Artificial Sequence Synthetic peptide
  MOD_RES (2): Sulfated Tyr
  MOD_RES (4): Sulfated Tyr
  MOD_RES (7): Sulfated Tyr
EYEYLDYDFL

SEQ ID NO: 71 - protein, 9 aa, Artificial Sequence
  Description of Artificial Sequence Synthetic peptide
  MOD_RES (2): Sulfated Tyr
  MOD_RES (4): Sulfated Tyr
  MOD_RES (7): Sulfated Tyr
EYEYLDYDF

SEQ ID NO: 72 - protein, 8 aa, Artificial Sequence
  Description of Artificial Sequence Synthetic peptide
  MOD_RES (2): Sulfated Tyr
  MOD_RES (4): Sulfated Tyr
  MOD_RES (7): Sulfated Tyr
EYEYLDYD

SEQ ID NO: 73 - protein, 12 aa, Artificial Sequence
  Description of Artificial Sequence Synthetic peptide
  MOD_RES (5): Sulfated Tyr
QATEYEYLDYDF

SEQ ID NO: 74 - protein, 11 aa, Artificial Sequence
  Description of Artificial Sequence Synthetic peptide
  MOD_RES (5): Sulfated Tyr
QATEYEYLDYD

SEQ ID NO: 75 - protein, 12 aa, Artificial Sequence
  Description of Artificial Sequence Synthetic peptide
  MOD_RES (4): Sulfated Tyr
ATEYEYLDYDFL

SEQ ID NO: 76 - protein, 11 aa, Artificial Sequence
  Description of Artificial Sequence Synthetic peptide
  MOD_RES (4): Sulfated Tyr
ATEYEYLDYDF

SEQ ID NO: 77 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 78 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 79 - protein, 10 aa, Artificial Sequence
  Description of Artificial Sequence Synthetic peptide
  MOD_RES (4): Sulfated Tyr
ATEYEYLDYD

SEQ ID NO: 80 - protein, 11 aa, Artificial Sequence
  Description of Artificial Sequence Synthetic peptide
  MOD_RES (3): Sulfated Tyr
TEYEYLDYDFL

SEQ ID NO: 81 - protein, 10 aa, Artificial Sequence
  Description of Artificial Sequence Synthetic peptide
  MOD_RES (3): Sulfated Tyr
TEYEYLDYDF

SEQ ID NO: 82 - protein, 9 aa, Artificial Sequence
  Description of Artificial Sequence Synthetic peptide
  MOD_RES (3): Sulfated Tyr
TEYEYLDYD

SEQ ID NO: 83 - protein, 10 aa, Artificial Sequence
  Description of Artificial Sequence Synthetic peptide
  MOD_RES (2): Sulfated Tyr
EYEYLDYDFL

SEQ ID NO: 84 - protein, 9 aa, Artificial Sequence
  Description of Artificial Sequence Synthetic peptide
  MOD_RES (2): Sulfated Tyr
EYEYLDYDF

SEQ ID NO: 85 - protein, 8 aa, Artificial Sequence
  Description of Artificial Sequence Synthetic peptide
  MOD_RES (2): Sulfated Tyr
EYEYLDYD

SEQ ID NO: 86 - protein, 12 aa, Artificial Sequence
  Description of Artificial Sequence Synthetic peptide
  MOD_RES (7): Sulfated Tyr
QATEYEYLDYDF

SEQ ID NO: 87 - protein, 11 aa, Artificial Sequence
  Description of Artificial Sequence Synthetic peptide
  MOD_RES (7): Sulfated Tyr
QATEYEYLDYD

SEQ ID NO: 88 - protein, 12 aa, Artificial Sequence
  Description of Artificial Sequence Synthetic peptide
  MOD_RES (6): Sulfated Tyr
ATEYEYLDYDFL

SEQ ID NO: 89 - protein, 11 aa, Artificial Sequence
  Description of Artificial Sequence Synthetic peptide
  MOD_RES (6): Sulfated Tyr
ATEYEYLDYDF

SEQ ID NO: 90 - protein, 10 aa, Artificial Sequence
  Description of Artificial Sequence Synthetic peptide
  MOD_RES (6): Sulfated Tyr
ATEYEYLDYD

SEQ ID NO: 91 - protein, 11 aa, Artificial Sequence
  Description of Artificial Sequence Synthetic peptide
  MOD_RES (5): Sulfated Tyr
TEYEYLDYDFL

SEQ ID NO: 92 - protein, 10 aa, Artificial Sequence
  Description of Artificial Sequence Synthetic peptide
  MOD_RES (5): Sulfated Tyr
TEYEYLDYDF

SEQ ID NO: 93 - protein, 9 aa, Artificial Sequence
  Description of Artificial Sequence Synthetic peptide
  MOD_RES (5): Sulfated Tyr
TEYEYLDYD

SEQ ID NO: 94 - protein, 10 aa, Artificial Sequence
  Description of Artificial Sequence Synthetic peptide
  MOD_RES (4): Sulfated Tyr
EYEYLDYDFL

SEQ ID NO: 95 - protein, 9 aa, Artificial Sequence
  Description of Artificial Sequence Synthetic peptide
  MOD_RES (4): Sulfated Tyr
EYEYLDYDF

SEQ ID NO: 96 - protein, 8 aa, Artificial Sequence
  Description of Artificial Sequence Synthetic peptide
  MOD_RES (4): Sulfated Tyr
EYEYLDYD

SEQ ID NO: 97 - protein, 12 aa, Artificial Sequence
  Description of Artificial Sequence Synthetic peptide
  MOD_RES (10): Sulfated Tyr
QATEYEYLDYDF

SEQ ID NO: 98 - protein, 11 aa, Artificial Sequence
  Description of Artificial Sequence Synthetic peptide
  MOD_RES (10): Sulfated Tyr
QATEYEYLDYD

SEQ ID NO: 99 - protein, 12 aa, Artificial Sequence
  Description of Artificial Sequence Synthetic peptide
  MOD_RES (9): Sulfated Tyr
ATEYEYLDYDFL

SEQ ID NO: 100 - protein, 11 aa, Artificial Sequence
  Description of Artificial Sequence Synthetic peptide
  MOD_RES (9): Sulfated Tyr
ATEYEYLDYDF

SEQ ID NO: 101 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 102 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 103 - protein, 10 aa, Artificial Sequence
  Description of Artificial Sequence Synthetic peptide
  MOD_RES (9): Sulfated Tyr
ATEYEYLDYD

SEQ ID NO: 104 - protein, 11 aa, Artificial Sequence
  Description of Artificial Sequence Synthetic peptide
  MOD_RES (8): Sulfated Tyr
TEYEYLDYDFL

SEQ ID NO: 105 - protein, 10 aa, Artificial Sequence
  Description of Artificial Sequence Synthetic peptide
  MOD_RES (8): Sulfated Tyr
TEYEYLDYDF

SEQ ID NO: 106 - protein, 9 aa, Artificial Sequence
  Description of Artificial Sequence Synthetic peptide
  MOD_RES (8): Sulfated Tyr
TEYEYLDYD

SEQ ID NO: 107 - protein, 10 aa, Artificial Sequence
  Description of Artificial Sequence Synthetic peptide
  MOD_RES (7): Sulfated Tyr
EYEYLDYDFL

SEQ ID NO: 108 - protein, 9 aa, Artificial Sequence
  Description of Artificial Sequence Synthetic peptide
  MOD_RES (7): Sulfated Tyr
EYEYLDYDF

SEQ ID NO: 109 - protein, 8 aa, Artificial Sequence
  Description of Artificial Sequence Synthetic peptide
  MOD_RES (7): Sulfated Tyr
EYEYLDYD

SEQ ID NO: 110 - protein, 12 aa, Artificial Sequence
  Description of Artificial Sequence Synthetic peptide
  MOD_RES (5): Sulfated Tyr
  MOD_RES (10): Sulfated Tyr
QATEYEYLDYDF

SEQ ID NO: 111 - protein, 11 aa, Artificial Sequence
  Description of Artificial Sequence Synthetic peptide
  MOD_RES (5): Sulfated Tyr
  MOD_RES (10): Sulfated Tyr
QATEYEYLDYD

SEQ ID NO: 112 - protein, 12 aa, Artificial Sequence
  Description of Artificial Sequence Synthetic peptide
  MOD_RES (4): Sulfated Tyr
  MOD_RES (9): Sulfated Tyr
ATEYEYLDYDFL

SEQ ID NO: 113 - protein, 11 aa, Artificial Sequence
  Description of Artificial Sequence Synthetic peptide
  MOD_RES (4): Sulfated Tyr
  MOD_RES (9): Sulfated Tyr
ATEYEYLDYDF

SEQ ID NO: 114 - protein, 10 aa, Artificial Sequence
  Description of Artificial Sequence Synthetic peptide
  MOD_RES (4): Sulfated Tyr
  MOD_RES (9): Sulfated Tyr
ATEYEYLDYD

SEQ ID NO: 115 - protein, 11 aa, Artificial Sequence
  Description of Artificial Sequence Synthetic peptide
  MOD_RES (3): Sulfated Tyr
  MOD_RES (8): Sulfated Tyr
TEYEYLDYDFL

SEQ ID NO: 116 - protein, 10 aa, Artificial Sequence
  Description of Artificial Sequence Synthetic peptide
  MOD_RES (3): Sulfated Tyr
  MOD_RES (8): Sulfated Tyr
TEYEYLDYDF

SEQ ID NO: 117 - protein, 9 aa, Artificial Sequence
  Description of Artificial Sequence Synthetic peptide
  MOD_RES (3): Sulfated Tyr
  MOD_RES (8): Sulfated Tyr
TEYEYLDYD

SEQ ID NO: 118 - protein, 10 aa, Artificial Sequence
  Description of Artificial Sequence Synthetic peptide
  MOD_RES (2): Sulfated Tyr
  MOD_RES (7): Sulfated Tyr
EYEYLDYDFL

SEQ ID NO: 119 - protein, 9 aa, Artificial Sequence
  Description of Artificial Sequence Synthetic peptide
  MOD_RES (2): Sulfated Tyr
  MOD_RES (7): Sulfated Tyr
EYEYLDYDF

SEQ ID NO: 120 - protein, 8 aa, Artificial Sequence
  Description of Artificial Sequence Synthetic peptide
  MOD_RES (2): Sulfated Tyr
  MOD_RES (7): Sulfated Tyr
EYEYLDYD

SEQ ID NO: 121 - protein, 12 aa, Artificial Sequence
  Description of Artificial Sequence Synthetic peptide
  MOD_RES (7): Sulfated Tyr
  MOD_RES (10): Sulfated Tyr
QATEYEYLDYDF

SEQ ID NO: 122 - protein, 11 aa, Artificial Sequence
  Description of Artificial Sequence Synthetic peptide
  MOD_RES (7): Sulfated Tyr
  MOD_RES (10): Sulfated Tyr
QATEYEYLDYD

SEQ ID NO: 123 - protein, 12 aa, Artificial Sequence
  Description of Artificial Sequence Synthetic peptide
  MOD_RES (6): Sulfated Tyr
  MOD_RES (9): Sulfated Tyr
ATEYEYLDYDFL

SEQ ID NO: 124 - protein, 11 aa, Artificial Sequence
  Description of Artificial Sequence Synthetic peptide
  MOD_RES (6): Sulfated Tyr
  MOD_RES (9): Sulfated Tyr
ATEYEYLDYDF

SEQ ID NO: 125 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 126 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 127 - protein, 10 aa, Artificial Sequence
  Description of Artificial Sequence Synthetic peptide
  MOD_RES (6): Sulfated Tyr
  MOD_RES (9): Sulfated Tyr
ATEYEYLDYD

SEQ ID NO: 128 - protein, 11 aa, Artificial Sequence
  Description of Artificial Sequence Synthetic peptide
  MOD_RES (5): Sulfated Tyr
  MOD_RES (8): Sulfated Tyr
TEYEYLDYDFL

SEQ ID NO: 129 - protein, 10 aa, Artificial Sequence
  Description of Artificial Sequence Synthetic peptide
  MOD_RES (5): Sulfated Tyr
  MOD_RES (8): Sulfated Tyr
TEYEYLDYDF

SEQ ID NO: 130 - protein, 9 aa, Artificial Sequence
  Description of Artificial Sequence Synthetic peptide
  MOD_RES (5): Sulfated Tyr
  MOD_RES (8): Sulfated Tyr
TEYEYLDYD

SEQ ID NO: 131 - protein, 10 aa, Artificial Sequence
  Description of Artificial Sequence Synthetic peptide
  MOD_RES (4): Sulfated Tyr
  MOD_RES (7): Sulfated Tyr
EYEYLDYDFL

SEQ ID NO: 132 - protein, 9 aa, Artificial Sequence
  Description of Artificial Sequence Synthetic peptide
  MOD_RES (4): Sulfated Tyr
  MOD_RES (7): Sulfated Tyr
EYEYLDYDF

SEQ ID NO: 133 - protein, 8 aa, Artificial Sequence
  Description of Artificial Sequence Synthetic peptide
  MOD_RES (4): Sulfated Tyr
  MOD_RES (7): Sulfated Tyr
EYEYLDYD

SEQ ID NO: 134 - protein, 12 aa, Artificial Sequence
  Description of Artificial Sequence Synthetic peptide
  MOD_RES (5): Sulfated Tyr
  MOD_RES (7): Sulfated Tyr
QATEYEYLDYDF

SEQ ID NO: 135 - protein, 11 aa, Artificial Sequence
  Description of Artificial Sequence Synthetic peptide
  MOD_RES (5): Sulfated Tyr
  MOD_RES (7): Sulfated Tyr
QATEYEYLDYD

SEQ ID NO: 136 - protein, 12 aa, Artificial Sequence
  Description of Artificial Sequence Synthetic peptide
  MOD_RES (4): Sulfated Tyr
  MOD_RES (6): Sulfated Tyr
ATEYEYLDYDFL

SEQ ID NO: 137 - protein, 11 aa, Artificial Sequence
  Description of Artificial Sequence Synthetic peptide
  MOD_RES (4): Sulfated Tyr
  MOD_RES (6): Sulfated Tyr
ATEYEYLDYDF

SEQ ID NO: 138 - protein, 10 aa, Artificial Sequence
  Description of Artificial Sequence Synthetic peptide
  MOD_RES (4): Sulfated Tyr
  MOD_RES (6): Sulfated Tyr
ATEYEYLDYD

SEQ ID NO: 139 - protein, 11 aa, Artificial Sequence
  Description of Artificial Sequence Synthetic peptide
  MOD_RES (3): Sulfated Tyr
  MOD_RES (5): Sulfated Tyr
TEYEYLDYDFL

SEQ ID NO: 140 - protein, 10 aa, Artificial Sequence
  Description of Artificial Sequence Synthetic peptide
  MOD_RES (3): Sulfated Tyr
  MOD_RES (5): Sulfated Tyr
TEYEYLDYDF

SEQ ID NO: 141 - protein, 9 aa, Artificial Sequence
  Description of Artificial Sequence Synthetic peptide
  MOD_RES (3): Sulfated Tyr
  MOD_RES (5): Sulfated Tyr
TEYEYLDYD

SEQ ID NO: 142 - protein, 10 aa, Artificial Sequence
  Description of Artificial Sequence Synthetic peptide
  MOD_RES (2): Sulfated Tyr
  MOD_RES (4): Sulfated Tyr
EYEYLDYDFL

SEQ ID NO: 143 - protein, 9 aa, Artificial Sequence
  Description of Artificial Sequence Synthetic peptide
  MOD_RES (2): Sulfated Tyr
  MOD_RES (4): Sulfated Tyr
EYEYLDYDF

SEQ ID NO: 144 - protein, 8 aa, Artificial Sequence
  Description of Artificial Sequence Synthetic peptide
  MOD_RES (2): Sulfated Tyr
  MOD_RES (4): Sulfated Tyr
EYEYLDYD

SEQ ID NO: 145 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 146 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 147 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 148 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 149 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 150 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 151 - protein, 13 aa, Artificial Sequence
  Description of Artificial Sequence Synthetic peptide
  MOD_RES (8): Sulfated Tyr
  MOD_RES (10): Sulfated Tyr
DDFEDPDYTYNTD

SEQ ID NO: 152 - protein, 13 aa, Artificial Sequence
  Description of Artificial Sequence Synthetic peptide
DDFEDPDYTYNTD

SEQ ID NO: 153 - protein, 13 aa, Artificial Sequence
  Description of Artificial Sequence Synthetic peptide
  MOD_RES (8): Sulfated Tyr
DDFEDPDYTYNTD

SEQ ID NO: 154 - protein, 13 aa, Artificial Sequence
  Description of Artificial Sequence Synthetic peptide
  MOD_RES (10): Sulfated Tyr
DDFEDPDYTYNTD

SEQ ID NO: 155 - protein, 13 aa, Artificial Sequence
  Description of Artificial Sequence Synthetic peptide
  MOD_RES (4): Sulfated Tyr
  MOD_RES (7): Sulfated Tyr
  MOD_RES (10): Sulfated Tyr
DFEYPDYSVYGTD

SEQ ID NO: 156 - protein, 13 aa, Artificial Sequence
  Description of Artificial Sequence Synthetic peptide
DFEYPDYSVYGTD

SEQ ID NO: 157 - protein, 13 aa, Artificial Sequence
  Description of Artificial Sequence Synthetic peptide
  MOD_RES (4): Sulfated Tyr
DFEYPDYSVYGTD

SEQ ID NO: 158 - protein, 13 aa, Artificial Sequence
  Description of Artificial Sequence Synthetic peptide
  MOD_RES (7): Sulfated Tyr
DFEYPDYSVYGTD

SEQ ID NO: 159 - protein, 13 aa, Artificial Sequence
  Description of Artificial Sequence Synthetic peptide
  MOD_RES (10): Sulfated Tyr
DFEYPDYSVYGTD

SEQ ID NO: 160 - protein, 13 aa, Artificial Sequence
  Description of Artificial Sequence Synthetic peptide
  MOD_RES (4): Sulfated Tyr
  MOD_RES (7): Sulfated Tyr
DFEYPDYSVYGTD

SEQ ID NO: 161 - protein, 13 aa, Artificial Sequence
  Description of Artificial Sequence Synthetic peptide
  MOD_RES (4): Sulfated Tyr
  MOD_RES (10): Sulfated Tyr
DFEYPDYSVYGTD

SEQ ID NO: 162 - protein, 13 aa, Artificial Sequence
  Description of Artificial Sequence Synthetic peptide
  MOD_RES (7): Sulfated Tyr
  MOD_RES (10): Sulfated Tyr
DFEYPDYSVYGTD

SEQ ID NO: 163 - protein, 13 aa, Artificial Sequence
  Description of Artificial Sequence Synthetic peptide
  MOD_RES (6): Sulfated Tyr
  MOD_RES (8)..(9): Sulfated Tyr
GDTDLYDYYPEED

SEQ ID NO: 164 - protein, 13 aa, Artificial Sequence
  Description of Artificial Sequence Synthetic peptide
GDTDLYDYYPEED

SEQ ID NO: 165 - protein, 13 aa, Artificial Sequence
  Description of Artificial Sequence Synthetic peptide
  MOD_RES (6): Sulfated Tyr
GDTDLYDYYPEED

SEQ ID NO: 166 - protein, 13 aa, Artificial Sequence
  Description of Artificial Sequence Synthetic peptide
  MOD_RES (8): Sulfated Tyr
GDTDLYDYYPEED

SEQ ID NO: 167 - protein, 13 aa, Artificial Sequence
  Description of Artificial Sequence Synthetic peptide
  MOD_RES (9): Sulfated Tyr
GDTDLYDYYPEED

SEQ ID NO: 168 - protein, 13 aa, Artificial Sequence
  Description of Artificial Sequence Synthetic peptide
  MOD_RES (6): Sulfated Tyr
  MOD_RES (8): Sulfated Tyr
GDTDLYDYYPEED

SEQ ID NO: 169 - protein, 13 aa, Artificial Sequence
  Description of Artificial Sequence Synthetic peptide
  MOD_RES (6): Sulfated Tyr
  MOD_RES (9): Sulfated Tyr
GDTDLYDYYPEED

SEQ ID NO: 170 - protein, 13 aa, Artificial Sequence
  Description of Artificial Sequence Synthetic peptide
  MOD_RES (8)..(9): Sulfated Tyr
GDTDLYDYYPEED

SEQ ID NO: 171 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 172 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 173 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 174 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 175 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
AATEYEYLDYDFL

SEQ ID NO: 176 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QAAEYEYLDYDFL

SEQ ID NO: 177 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATAYEYLDYDFL

SEQ ID NO: 178 - protein, 13 aa, Artificial Sequence
  Description of Artificial Sequence Synthetic peptide
  MOD_RES (7): Sulfated Tyr
  MOD_RES (10): Sulfated Tyr
QATEAEYLDYDFL

SEQ ID NO: 179 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYAYLDYDFL

SEQ ID NO: 180 - protein, 13 aa, Artificial Sequence
  Description of Artificial Sequence Synthetic peptide
  MOD_RES (5): Sulfated Tyr
  MOD_RES (10): Sulfated Tyr
QATEYEALDYDFL

SEQ ID NO: 181 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYADYDFL

SEQ ID NO: 182 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLAYDFL

SEQ ID NO: 183 - protein, 13 aa, Artificial Sequence
  Description of Artificial Sequence Synthetic peptide
  MOD_RES (5): Sulfated Tyr
  MOD_RES (7): Sulfated Tyr
QATEYEYLDADFL

SEQ ID NO: 184 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYAFL

SEQ ID NO: 185 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDAL

SEQ ID NO: 186 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFA

SEQ ID NO: 187 - protein, 13 aa, Artificial Sequence
  Description of Artificial Sequence Synthetic peptide
QATEYEYLDYDFL

SEQ ID NO: 188 - protein, 13 aa, Artificial Sequence
  Description of Artificial Sequence Synthetic peptide
  MOD_RES (5): Sulfated Tyr
QATEYEYLDYDFL

SEQ ID NO: 189 - protein, 13 aa, Artificial Sequence
  Description of Artificial Sequence Synthetic peptide
  MOD_RES (7): Sulfated Tyr
QATEYEYLDYDFL

SEQ ID NO: 190 - protein, 13 aa, Artificial Sequence
  Description of Artificial Sequence Synthetic peptide
  MOD_RES (10): Sulfated Tyr
QATEYEYLDYDFL

SEQ ID NO: 191 - protein, 13 aa, Artificial Sequence
  Description of Artificial Sequence Synthetic peptide
  MOD_RES (5): Sulfated Tyr
  MOD_RES (7): Sulfated Tyr
QATEYEYLDYDFL

SEQ ID NO: 192 - protein, 13 aa, Artificial Sequence
  Description of Artificial Sequence Synthetic peptide
  MOD_RES (5): Sulfated Tyr
  MOD_RES (10): Sulfated Tyr
QATEYEYLDYDFL

SEQ ID NO: 193 - protein, 13 aa, Artificial Sequence
  Description of Artificial Sequence Synthetic peptide
  MOD_RES (7): Sulfated Tyr
  MOD_RES (10): Sulfated Tyr
QATEYEYLDYDFL

SEQ ID NO: 194 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 195 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 196 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 197 - protein, 13 aa, Artificial Sequence
  Description of Artificial Sequence Synthetic peptide
  MOD_RES (5): Sulfated Tyr
  MOD_RES (7): Sulfated Tyr
  MOD_RES (10): Sulfated Tyr
QATEYEYLDYDFL

SEQ ID NO: 198 - protein, 446 aa, Homo sapiens
QVQLVQSGAEVKKPGSSVRVSCKASGGSFSSYGIHWVQQAPGQGLEWMGRIIPVLRIRNYAQKFHDRVTIDADTSTGTAYMELSSLTSDDTAVYYCAGSRQGVAPTGYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 199 - protein, 221 aa, Homo sapiens
QAVLTQPSSLSASPGASASLTCNLRSGIDVGTYRIYWYQQKPGSPPQYLLRYKSDSDKQQGSGVPSRFSGTKDASANAGILLISGLQSEDEADYYCMIWHSNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 200 - protein, 447 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKQDGNWGRYFDYWGRGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 201 - protein, 199 aa, Homo sapiens
QTVRITCQGDSLRSYYASWYQQKAGQAPVVVVFGKNKRPSGLPDRFSGSSSGNTASLTITGAQAEDEADYYCSSRDSSGKRRVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 202 - protein, 442 aa, Homo sapiens
EVQLLESGGGLVRPGGSLRLSCAASGFTFSSYAMSWVRQAPGKGLEWVSAISGSGGSTYYADSVKGRFTISRDNSKNTLYLQMNSLRAEDTAVYYCARDKRLTYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 203 - protein, 216 aa, Homo sapiens
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSARVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 204 - protein, 15 aa, Artificial Sequence
  Description of Artificial Sequence Synthetic linker peptide
GGGGSGGGGSGGGGS

SEQ ID NO: 205 - DNA, 780 bp, Homo sapiens
caggtccagctggtgcagtctggggctgaggtgaagaagcctgggtcctcggtgagggtctcctgcaaggcttctggaggctccttcagcagctatggtatccactgggtgcgacaggcccctggtcaagggcttgagtggatggggaggatcatccctgtccttcgtataagaaactacgcacagaagttccacgacagagtcaccataaccgcggacacatccacgagcacagcctacatggaattgagcagcctgacatctgacgacacggccgtgtattactgtgcgggttctcggcaaggcgttgccccgactggctactggggccaggggacaatggtcaccgtctcgagtggaggcggcggttcaggcggaggtggctctggcggtggcggaagtgcacaggctgtgctgactcagccggcttccctctctgcatctcctggagcatcagccagtctcacctgcaacttacgcagtggcatcgatgttggtacctacaggatatactggtaccagcagaagccagggagtcctccccagtatctcctgaggtacaaatcagactcagataagcagcagggctctggagtccccagccgcttctctggatccaaggatgcttcggccaatgcagggattttactcatctctgggctccagtctgaggatgaggctgactattactgtatgatttggcacagcaacggttgggtgttcggcggagggaccaagctgaccgtcctaggtgcggccgcacatcatcatcaccatcat

SEQ ID NO: 206 - protein, 260 aa, Homo sapiens
QVQLVQSGAEVKKPGSSVRVSCKASGGSFSSYGIHWVRQAPGQGLEWMGRIIPVLRIRNYAQKFHDRVTITADTSTSTAYMELSSLTSDDTAVYYCAGSRQGVAPTGYWGQGTMVTVSSGGGGSGGGGSGGGGSAQAVLTQPASLSASPGASASLTCNLRSGIDVGTYRIYWYQQKPGSPPQYLLRYKSDSDKQQGSGVPSRFSGSKDASANAGILLISGLQSEDEADYYCMIWHSNGWVFGGGTKLTVLGAAAHHHHHH

SEQ ID NO: 207 - protein, 446 aa, Homo sapiens
QVQLVQSGAEVKKPGSSVRVSCKASGGSFSSYGIHWVRQAPGQGLEWMGRIIPVLRIRNYAQKFHDRVTITADTSTSTAYMELSSLTSDDTAVYYCAGSRQGVAPTGYWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 208 - protein, 221 aa, Homo sapiens
QAVLTQPASLSASPGASASLTCNLRSGIDVGTYRIYWYQQKPGSPPQYLLRYKSDSDKQQGSGVPSRFSGSKDASANAGILLISGLQSEDEADYYCMIWHSNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2007/0160601 A1
(43) Pub. Date: Jul. 12, 2007

(54) NEUTRALIZING ANTIBODIES AGAINST PRIMATE PSGL-1 AND USES THEREFOR

(76) Inventors: Angela Widom, Acton, MA (US); Kimberly A. Marquette, Somerville, MA (US); Gray D. Shaw, Milton, MA (US); Louise A. Conroy, Cambridge (GB); David C. Lowe, Hertfordshire (GB)

Correspondence Address: WYETH/FINNEGAN HENDERSON, LLP, 901 NEW YORK AVENUE, NW, WASHINGTON, DC, 20001-4413, US

(21) Appl. No.: 11/608,627

(22) Filed: Dec. 8, 2006

Related U.S. Application Data
(60) Provisional application No. 60/748,984, filed on Dec. 9, 2005.

(51) Int. Cl. A61K 39/395; C07H 21/04; C12P 21/06; C12N 5/06; C07K 16/28

(52) U.S. Cl. 424/143.1; 530/388.22; 435/69.1; 435/320.1; 435/334; 536/23.53

(57) ABSTRACT

This application relates to neutralizing antibodies that specifically bind primate PSGL-1, as well as their production and use. The antibodies reduce one or more activities of PSGL-1, such as human PSGL-1. Methods to detect or quantitate PSGL-1 in a biological sample by adding an antibody that specifically binds to PSGL-1 to the sample are provided. Further, methods to treat a primate PSGL-1 associated disorder, such as a human disorder, by administering a PSGL-1 specific antibody are also provided.

### RELATED APPLICATIONS

This application claims the benefit of U.S. Provisional Application No. 60/748,984, filed on Dec. 9, 2005, the contents of which are incorporated herein in their entirety by reference.

### BACKGROUND

The selectins are a family of calcium-dependent type I membrane glycoproteins that play a significant role in the regulation of cell adhesion and cell signaling in immune and inflammatory responses. The selectin family includes three members that display different patterns of expression and function. L-selectin is constitutively expressed by the majority of circulating leukocytes, and is implicated in homing and leukocyte recruitment. L-selectin can be shed from cell surfaces upon activation. P-selectin (also known as CD62, GMP-140, and PAD-GEM) is stored in Weibel-Palade bodies of resting endothelial cells and in alpha granules of unstimulated platelets, and is rapidly translocated to the cell surface in activated endothelium and activated platelets, where it promotes the tethering and rapid rolling of leukocytes. E-selectin (also known as ELAM-1) is expressed by activated endothelium and is involved in the slow rolling of leukocytes.

The selectins are type I membrane glycoproteins. The extracellular N-terminal domain of these proteins contains an N-terminal cytoplasmic C-type lectin domain, an EGF-like domain, and a series of short consensus repeats. The C-terminal cytoplasmic domain is short.

E-, L-, and P-selectins bind selectively, but with low affinity, to certain oligosaccharides such as sialyl Lewis x (sLe^{x}) and sialyl Lewis a (sLe^{a}). L- and P-selectins bind to heparan sulfate. The selectins bind with greater affinity or avidity to mucin-type glycoproteins having multiple O-linked glycans and repeating peptide motifs (McEver et al., J. Clin. Invest. 100:485-492 (1997)).

P-selectin glycoprotein ligand-1 (PSGL-1; also known as CD162) is a leukocyte adhesion molecule that mediates cell tethering and rolling on activated endothelium cells under physiological blood flow. This activity is an important initial step in leukocyte extravasation. PSGL-1 was initially identified as a ligand for P-selectin, and subsequent work has revealed that PSGL-1 is also a ligand for E-selectin and L-selectin (see, e.g., U.S. Pat. No. 6,277,975).

PSGL-1 is a mucin-like, homodimeric, disulfide-bonded, glycoprotein that is expressed on the surface of most hematopoietic cells, including, e.g., neutrophils, monocytes, lymphocytes, dendritic cells, and platelets. Human PSGL-1 has an amino terminal signal peptide (amino acid residues 1-18) and a propeptide (amino acid residues 19-41) with a consensus cleavage site for paired basic amino acid converting enzymes (PACE). The N-terminal extracellular region of the mature protein begins at residue 42. The extracellular domain of the PSGL-1 molecule further contains several serine/threonine rich decameric repeats containing multiple O-glycosylation linkage sites. This region of the molecule, which folds into a rod-like structure, is responsible for the mucin-like characteristics of PSGL-1. On neutrophils, this rod-like structure and the localization of PSGL-1 on the tips of microvilli facilitates the binding of PSGL-1 to selectin-expressing cells. The decameric repeat region of PSGL-1 is followed by the transmembrane region (residues 268-292) and the cytoplasmic domain (residues293-361).

Murine PSGL-1 is similar in size to human PSGL-1, and also has a signal peptide and a propeptide. However, murine PSGL-1 has two, rather than three, tyrosine residues at its anionic N-terminus. The transmembrane and cytoplasmic domains are the most highly conserved sequences between murine and human PSGL-1, suggesting an important conserved function(s) for those domains.

The mature amino terminus of PSGL-1 has an anionic segment (the amino-terminal 19 amino acids, i.e., residues 42-61), with several sulfated tyrosines that are critical for binding to P-selectin and L-selectin. The amino acid context of the sulfated tyrosines is substantially different in rat, mouse, and human PSGL-1, as they are located within different primary amino acid sequences.

Although most lymphocytes express PSGL-1, only those lymphocytes having certain post-translational modifications interact with the selectins (Frenette et al., J. Exp. Med. 191:1413-1422 (2000)). High affinity interaction of PSGL-1 with P-selectin requires sulfation of tyrosines, e.g., at residues 46, 48, and 51 (human) or 54 and 56 (mouse) (Sako et al., Cell 83:323-331 (1995), Xia et al., Blood 101:552-559 (2003)). Sulfation of at least one tyrosine is required for binding. High affinity interaction of PSGL-1 with P-selectin also requires O-linked glycosylation of Thr-16 with sialylated, fucosylated, core 2 O-glycans. However, N-linked glycosylation is not essential for binding to P-selectin. N-linked glycans can be enzymatically removed and N-linked glycosylation sites can be removed by mutation without affecting binding (McEver et al., J. Clin. Invest. 100:485-492(1997)).

Binding of PSGL-1 to L-selectin also requires tyrosine sulfation and O-glycosylation in the N-terminal region, although it is not known whether the same sulfation and glycosylation patterns are recognized by L- and P-selectins. By contrast, E-selectin binding to PSGL-1 requires sialylated, fucosylated core-2 O-linked glycans, but does not require tyrosine sulfation. E-selectin binds to the N-terminal region of PSGL-1 with low affinity, but may also bind to other, uncharacterized, binding sites (McEver et al., J. Clin. Invest. 100:485-492 (1997)).

In addition to interacting with selectins, PSGL-1 also plays a role in signal transduction. It has been reported that the cytoplasmic tail of PSGL-1 interacts with cytoskeleton linkers, such as ezrin and moesin. Proteins of the ezrin/radixin/moesin (ERM) family function as membrane-actin cytoskeleton linkers and play a key role in the formation of protrusive plasma membrane structures. Furthermore, the engagement of PSGL-1 with either P-selectin or anti-PSGL-1 antibodies induces tyrosine phosphorylation, activation of MAP kinases in human neutrophils, and cytokine (e.g., IL-8) release by neutrophils, monocytes, and T cells. Further, soluble forms of P-selectin can promote the generation of procoagulant, leukocyte-derived microparticles or microvesicles and normalize bleeding time in hemophilia A mice. This activity is mediated by PSGL-1 (see, e.g., Hrachovinova et al., Nature Med. 9:1020-1025 (2003); Cambien et al., Trends. Mol. Med. 10:179-186 (2004)).

A number of antibodies to PSGL-1 have been reported (see, e.g., Moore et al., J. Cell Biol. 128:661-671 (1995); Snapp et al., Blood 91:154-164 (1998); U.S. Pat. Nos. 5,852,175; 6,277,975 B1; and U.S. Patent Application Pub. Nos. 2004/0116333 A1; 2005/0130206 A1; 2005/0152906 A1). For example, antibodies that bind to human PSGL-1 and block the interaction of PSGL-1 with P-selectin are described in Thatte et al., J. Leuk. Biol, 72:470-477 (2002). The binding of these antibodies to human PSGL-1 does not require tyrosine sulfate modification of the PSGL-1 molecule. Some of these antibodies are commercially available, e.g., the mouse anti-human monoclonal antibody TB5 (EXBIO Praha, Czech Republic), and the mouse antibody PL1 (Ancell Immunology Research Products, Bayport, Minn.; Research Diagnostics Inc., Concord, Mass.; EMD Biosciences, San Diego, Calif.), but further antibodies that inhibit the sulfotyrosine-mediated interaction of P-selectin and PSGL-1 as well as information about their in vivo and in vitro effects are needed.

There is a need for antibodies that bind PSGL-1, including antibodies that are specific for human and/or primate PSGL-1 proteins, including sulfated forms, with high affinity and high specificity for research, diagnostic, and therapeutic uses.

### SUMMARY

This application relates to PSGL-1 specific antibodies that are capable of binding to a primate PSGL-1, as well as their production and use. The antibodies described herein are specific for primate, including human, PSGL-1. They may also specifically bind to sulfated PSGL-1 as compared to unsulfated PSGL-1 and in some embodiments, are capable of inhibiting prothrombotic activity. In certain embodiments, the antibody comprises an amino acid sequence chosen from SEQ ID NO:2, SEQ ID NO:4, SEQ ID NO:6, SEQ ID NO:8, SEQ ID NO:10, SEQ ID NO:12, SEQ ID NO:14, SEQ ID NO:16 and SEQ ID NO:18, wherein the antibody is capable of specifically binding to a primate PSGL-1. In other embodiments the antibody comprises a CDR region of these antibodies, i.e., an amino acid sequence chosen from SEQ ID NOs:19-36. Monoclonal, human, and scFv antibodies are specifically contemplated, as are antibodies that specifically bind with an affinity constant greater than 10⁸ M⁻¹. In some embodiments, the antibody specifically binds to EYEYLDyDF (SEQ ID NO:45).

In some embodiments, the antibodies comprise an Fc domain with altered effector function. For example, the antibodies may comprise an Fc portion of an antibody with specific amino acid substitutions to diminish Fc-mediated effector function of the antibody.

Nonlimiting illustrative embodiments of the antibodies are referred to as PSG3, PSG5, and PSG6. Other embodiments comprise a V_{H} and/or V_{L} domain of the Fv fragment of PSG3, PSG5, or PSG6, or an scFv containing both the V_{H} and V_{L} domains. Further embodiments comprise one or more complementarity determining regions (CDRs) of any of these V_{H} and V_{L} domains. In particular embodiments the antibodies comprise an H3 fragment of the V_{H} domain of PSG3, PSG5, or PSG6. Compositions comprising primate PSGL-1 specific antibodies, and their use, are also provided.

In another aspect, the invention includes isolated nucleic acids which comprise a sequence encoding an antibody described herein. Another aspect provides an isolated nucleic acid, which comprises a sequence encoding a V_{H} or V_{L} domain from an Fv fragment of PSG3, PSG5, or PSG6, or that comprises a sequence encoding an scFv with both the V_{H} and V_{L} domains. An isolated nucleic acid, which comprises a sequence encoding at least one CDR from any of the presently disclosed V_{H} and V_{L} domains, is also disclosed. Another aspect provides DNA constructs and host cells comprising such a nucleic acid.

Yet another aspect provides a method of producing V_{H} and V_{L} domains and/or functional antibodies comprising all or a portion of such domains derived from the V_{H} or V_{L} domains of PSG3, PSG5, or PSG6.

In another aspect, the disclosure provides methods to identify and quantify primate PSGL-1 proteins in a biological sample such as, e.g., human PSGL-1 and its fragments. In particular embodiments, the PSGL-1 specific antibodies are used in a biomarker assay to detect PSGL-1 proteins in a biological sample.

In one embodiment, the presently disclosed antibodies may be used as a diagnostic tool to quantitatively or qualitatively detect a primate or human PSGL-1 or its fragments in a biological sample, which may be, for example, from an individual having or suspected of having a PSGL-1 associated disorder. The presence or amount of primate PSGL-1 detected can be correlated with the expression and/or post-translational modification (e.g., sulfation) of PSGL-1.

Other aspects provide compositions comprising antibodies of the invention or their antigen-binding fragments, and their use in methods of inhibiting or neutralizing PSGL-1, including methods of treating a PSGL-1 associated disorder in an animal, including a mammal such as a primate or a human. The disclosure provides methods to treat or prevent conditions in which a reduction in inflammation is desirable. For example, the presently disclosed antibodies may be used in therapies to treat or prevent disorders associated with PSGL-1, leukocyte adhesion and/or movement, tumor metastasis, atherosclerosis, cardiovascular disorders, and autoimmune diseases.

It is to be understood that both the foregoing general description and the following detailed description are exemplary and explanatory only and are not restrictive of the claimed invention.

### BRIEF DESCRIPTION OF THE FIGURES

FIG. 1 shows the DNA sequence of PSG3 scFv (SEQ ID NO:1) in FIG. 1(A); the amino acid sequence of PSG3 scFv (SEQ ID NO:2) in FIG. 1(B), the V_{H} region in bold (SEQ ID NO:4) and the V_{L} region in bold underline (SEQ ID NO:6); the amino acid sequence of the V_{H} region (SEQ ID NO:4) linked to a human IgG4 sequence (SEQ ID NO:39) in FIG. 1(C) (SEQ ID NO:198); and the V_{L} region (SEQ ID NO:6) linked to a human lambda sequence (SEQ ID NO:40) in FIG. 1(D) (SEQ ID NO:199). FIG. 1(E) shows the amino acid sequence of PSG3 G1 (SEQ ID NO:37), which links the V_{H} region of PSG3 with a human IgG1 Fc region that has reduced effector function. Underlined amino acids differ from the wild-type IgG1 Fc sequences. FIG. 1(G)-FIG. 1(I) show partially germlined PSG3 sequences. Variable region sequences are indicated in bold; the V_{H} region is shown in bold, and the V_{L} region is shown in bold underline in FIGS. 1(A) and (B).

FIG. 2 shows the DNA sequence of PSG5 scFv (SEQ ID NO:7) in FIG. 2(A); the amino acid sequence of PSG5 scFv (SEQ ID NO:8) in FIG. 2(B), the V_{H} region (SEQ ID NO:10) in bold and the V_{L} region (SEQ ID NO:12) in bold underline; the amino acid sequence of the V_{H} region (SEQ ID NO:10) linked to a human IgG4 sequence (SEQ ID NO:39) in FIG. 2(C) (SEQ ID NO:200); and the V_{L} region (SEQ ID NO:12) linked to a human lambda sequence (SEQ ID NO:40) in FIG. 2(D) (SEQ ID NO:201). Variable region sequences are indicated as in FIG. 1.

FIG. 3 shows the DNA sequence of PSG6 scFv (SEQ ID NO:13) in FIG. 3(A); the amino acid sequence of PSG6 scFv (SEQ ID NO:14) in FIG. 3(B), the V_{H} region (SEQ ID NO:16) in bold and the V_{L} region (SEQ ID NO:18) in bold underline; the amino acid sequence of the V_{H} region (SEQ ID NO:16) linked to a human IgG4 sequence (SEQ ID NO:39) in FIG. 3(C) (SEQ ID NO:202); and the V_{L} region (SEQ ID NO:18) linked to a human lambda sequence (SEQ ID NO:40) in FIG. 3(D) (SEQ ID NO:203). Variable region sequences are indicated as in FIG. 1.

FIG. 4 shows a competitive binding assay using a biotinylated human PSGL-1 19.ek.Fc fusion protein (FIG. 4(A)), and a biotinylated rPSGL Ig fusion protein (FIG. 4(B)). Representative results for PSG5 and PSG6 are shown.

FIG. 5 shows the results of a BIAcore binding assay using bivalent forms of the PSG3, PSG5, and PSG6 antibodies, indicating that PSG3, PSG5, and PSG6 specifically bind to a sulfated glycopeptide, 19.ek, derived from the sequence of PSGL-1 (QATEyEyLDyDFLPETEPPRPMMDDDDK (SEQ ID NO:42)), but not to forms of the peptide without sulfate-modified tyrosine residues, regardless of whether an O-linked glycan is present (FIG. 5(A)). The KPL-1 antibody specifically binds to the peptide, regardless of sulfation or glycosylation, and acts as a positive control. The 3D1 antibody which is of a similar isotype to the PSG3, PSG5, and PSG6 antibodies, binds an unrelated protein and serves as a negative control. FIG. 5(B) shows binding of the antibodies to the peptide with various degrees of sulfation.

FIG. 6 shows a cell adhesion assay in which HL-60 cells are added to a P-selectin coated plate in the presence of a range of antibody concentrations. Data are shown for PSG5 and PSG6, and for control antibodies PSG 4H10 and KPL-1, and are indicated as relative fluorescence units.

FIG. 7 shows the results of epitope mapping of the PSG3 antibody. FIG. 7(A) evaluates binding of the PSG3 antibody to peptides that vary from the phagemid library panning peptide, as set forth in Table 4. FIG. 7(B) shows a substitution analysis of a EYEYLDyDF (SEQ ID NO:45) peptide (where “y” is sulfated tyrosine and “Y” is non-sulfated tyrosine). The unmodified SEQ ID NO:45 peptide appears in the first and last pairs of columns and the top and bottom rows.

FIG. 8 shows the effect of a PSG3 antibody on thrombolysis in a non-human primate thrombosis model. In FIG. 8(A), the outline and timeline of the experimental procedure is shown. In FIG. 8(B) the average acceleration of time to clot lysis with the combination of a thrombolytic agent (Tenecteplase) and PSG3 antibody is shown. In FIG. 8(C) the improvement in the average time of vessel patency for the combination of thrombolytic and PSG3 antibody is shown.

### DETAILED DESCRIPTION

The invention comprises antibodies and fragments thereof that specifically bind to a primate PSGL-1 and reduce one or more biological activities of the PSGL-1. Also provided are novel human anti-P L-1 antibodies, termed PSG3, PSG5, and PSG6, and antibodies and antigen-binding fragments derived therefrom. As described herein, these antibodies were identified and isolated by using a PSGL-1 polypeptide as a “panning” reagent to identify single chain Fv fragments (scFv's) from human phage display libraries. These antibodies bind specifically to a sulfated fragment of primate PSGL-1, including non-human primate and/or human PSGL-1 and reduce the binding of PSGL-1 to P-selectin, L-selectin and/or E-selectin, for example.

The antibodies of the invention can be used to detect or quantitate the presence of human PSGL-1 and its fragments, for example. In addition, the antibodies can be used to study the biological functions of PSGL-1. Thus, the antibodies provide a useful tool for the study of leukocyte recruitment, inflammation, thrombosis, coagulation, and signaling cascades in vitro and in vivo. Methods for treating PSGL-1 associated disorders using the antibodies described herein are also provided. The antibodies of the invention possess a number of useful properties. The disclosed antibodies inhibit one or more PSGL-1 activities. In vitro and in vivo assays for PSGL-1 activity include, for example, assays measuring leukocyte adhesion, leukocyte rolling, e.g., by intravital microscopy, binding to P-selectin, L-selectin, and/or E-selectin, binding of PSGL-1 or its binding partner, e.g., P-selectin, to leukocytes, such as, neutrophils, as well as assays for inflammation, tumor cell adhesion, platelet aggregation; thrombosis, thrombolysis, coagulation, and leukostasis.

In order that the present invention may be more readily understood, certain terms are first defined. Additional definitions are set forth throughout the detailed description.

“Affinity tag,” as used herein, means a molecule attached to a second molecule of interest, capable of interacting with a specific binding partner for the purpose of isolating or identifying the second molecule of interest.

The term “antibody,” as used herein, refers to immunoglobulin molecules and immunologically active portions of immunoglobulin molecules, i.e., molecules that contain an antigen binding site that specifically binds (immunoreacts with) an antigen, such as a sulfated tyrosine or a polypeptide comprising a sulfated tyrosine. The term “antibody” encompasses any polypeptide comprising an antigen-binding site of an immunoglobulin regardless of the source, species of origin, method of production, and characteristics. As a non-limiting example, the term “antibody” includes human, orangutan, monkey, primate, mouse, rat, goat, sheep, and chicken antibodies. The term includes but is not limited to polyclonal, monoclonal, human, humanized, single-chain, chimeric, synthetic, recombinant, hybrid, mutated, resurfaced, and CDR-grafted antibodies. For the purposes of the present invention, it also includes, unless otherwise stated, antibody fragments such as Fab, F(ab′)₂, Fv, scFv, Fd, dAb, and other antibody fragments that retain the antigen-binding function. A “monoclonal antibody,” as used herein, refers to a population of antibody molecules that contain a particular antigen binding site and are capable of specifically binding to a particular epitope.

Antibodies can be made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibody Engineering, 2^{nd} ed., Borrebaeck, Ed., Oxford University Press, 1995; Antibodies: A Laboratory Manual, Harlow et al., Eds., Cold Spring Harbor Laboratory, 1988. An antibody optionally comprises a heterologous sequence such as an affinity tag, for example.

The term “antigen-binding domain” refers to the part of an antibody molecule that comprises the area specifically binding to or complementary to a part or all of an antigen. Where an antigen is large, for example, an antibody may only bind to a particular part of the antigen. The “epitope” or “antigenic determinant” is a portion of an antigen molecule that is responsible for specific interactions with the antigen-binding domain of an antibody. An antigen-binding domain may be provided by one or more antibody variable domains (e.g., a so-called Fd antibody fragment consisting of a V_{H} domain). An antigen-binding domain comprises an antibody light chain variable region (V_{L}) and an antibody heavy chain variable region (V_{H}).

“Bioavailability,” as used herein, means the extent and rate at which a substance is absorbed into a living system or is made available at the site of physiological activity.

A “biological sample” is biological material collected from cells, tissues, organs, or organisms. Exemplary biological samples include serum, blood, plasma, biopsy sample, tissue sample, cell suspension, biological fluid, saliva, oral fluid, cerebrospinal fluid, amniotic fluid, milk, colostrum, mammary gland secretion, lymph, urine, sweat, lacrimal fluid, gastric fluid, synovial fluid, mucus, and other samples and clinical specimens. A sample may be from a human, primate, non-human primate, mammal, or other animal, for example.

The term “DNA construct,” as used herein, means a DNA molecule, or a clone of such a molecule, either single- or double-stranded that has been modified to contain segments of DNA combined in a manner that as a whole would not otherwise exist in nature. DNA constructs contain the information necessary to direct the expression of polypeptides of interest. DNA constructs can include promoters, enhancers and transcription terminators. DNA constructs containing the information necessary to direct the secretion of a polypeptide will also contain at least one secretory signal sequence.

The term “effective dose,” or “effective amount,” refers to a dosage or level that is sufficient to ameliorate clinical symptoms of, or achieve a desired biological outcome (e.g., reduction in a systemic or localized inflammatory response, decreased coagulation, or increased fibrinolytic activity) in individuals, including individuals having a PSGL-1 associated disorder. Such amount should be sufficient to reduce one or more symptoms or manifestations of the disorder. Therapeutic outcomes and clinical symptoms may include, for example, a reduction in one or more symptoms of a systemic or localized inflammatory response such as, e.g., fever, delirium, chills, shaking, hypothermia, hyperventilation, or a rapid heartbeat, decreased coagulation, or a decreased leukocyte count. In one embodiment, a PSGL-1 specific antibody reduces clinical manifestations of an inflammatory, T cell mediated, coagulation or thrombotic associated disorder. In another embodiment, clinical manifestations of an immune or cardiovascular disorder, including the PSGL-1 associated disorders listed infra, are deduced. A PSGL-1 specific antibody can cause a decrease in measured levels of pro-inflammatory cytokines, for example. The effective amount can be determined as described in the subsequent sections. A “therapeutically effective amount” of a sulfotyrosine specific antibody refers to an amount which is effective, upon single or multiple dose administration to an individual (such as a human) to treat, prevent, cure, delay, reduce the severity of, or ameliorate at least one symptom of a disorder or recurring disorder, or to prolong the survival of the subject beyond that expected in the absence of such treatment.

A “fragment,” as used herein, refers to a portion of a polypeptide or nucleic acid, such as a sequence of at least 5 contiguous residues, of at least 10 contiguous residues, of at least 15 contiguous residues, of at least 20 contiguous residues, of at least 25 contiguous residues, of at least 40 contiguous residues, of at least 50 contiguous residues, of at least 100 contiguous residues, or of at least 200 contiguous residues, that retains activity of the original protein. Fragments with a length of approximately 5, 10, 15, 20, 25, 30, 40, 50, 100, 200 residues, or more are contemplated, for example.

A protein or peptide “homolog,” as used herein, means that a relevant amino acid sequence of a protein or a peptide is at least 70%, 75%, 80%, 85%, 90%, 95%, 96%, 97%, 98%, 99%, or 100% identical to a given sequence. By way of example, such sequences may be variants derived from various species, or the homologous sequence may be recombinantly produced. The sequence may be derived from the given sequence by truncation, deletion, amino acid substitution, or addition. Percent identity between two amino acid sequences is determined by standard alignment algorithms such as, for example, Basic Local Alignment Tool (BLAST) described in Altschul et al., J. Mol. Biol. 215:403-410 (1990). See also the algorithm of Needleman et al., J. Mol. Biol. 48:444-453 (1970); the algorithm of Meyers et al., Comput. Appl. Biosci. 4:11-17 (1988); or Tatusova et al., FEMS Microbiol. Lett. 174:247-250 (1999), and other alignment algorithms and methods of the art.

The term “individual” refers to any vertebrate animal, including a mammal, bird, reptile, amphibian, or fish. The term “mammal” includes any animal classified as such, male or female, including humans, non-human primates, primates, chimpanzees, gorillas, orangutans, monkeys, dogs, horses, cats, rats, mice, guinea pigs, etc. The term “primate” refers to humans, monkeys, and apes, for example. Examples of non-mammalian animals include frog, chicken, turkey, duck, goose, fish, salmon, catfish, bass, and trout.

The term “isolated” refers to a molecule that is substantially free of its natural environment. For instance, an isolated protein is substantially free of cellular material or other proteins from the cell or tissue source from which it was derived. The term also refers to preparations where the isolated protein is at least 70-80% (w/w) pure; or at least 80-90% (w/w) pure; or at least 90-95% pure; or at least 80%, 85%, 90%, 95%, 96%, 97%, 98%, 99%, or 100% (w/w) pure. In some embodiments, the isolated molecule is sufficiently pure for pharmaceutical compositions.

“Linked,” as used herein, refers to a first nucleic acid sequence covalently joined to a second nucleic acid sequence. The first nucleic acid sequence can be directly joined or juxtaposed to the second nucleic acid sequence, or alternatively an intervening moiety, such as a linker sequence, can covalently join the first sequence to the second sequence. Linked as used herein can also refer to a first amino acid sequence covalently joined to a second amino acid sequence, as above.

The terms “neutralize,” “neutralizing,” “inhibitory,” and their cognates refer to a reduction in an activity of PSGL-1 by a PSGL-1 inhibitor, relative to the activity of PSGL-1 in the absence of the same inhibitor. A neutralizing antibody may reduce one or more PSGL-1 activities. The reduction in activity is preferably at least about 10%, 20%, 30%, 40%, 50%, 60%, 70%, 75%, 80%, 85%, 90%, 95%, or higher.

“Operatively linked,” as used herein, means a first nucleic acid sequence linked to a second nucleic acid sequence such that both sequences are capable of being expressed as a biologically active protein or peptide.

The term “PSGL-1 activity” refers to biological activity associated with a PSGL-1 protein, such as leukocyte rolling, e.g., in post-capillary venules; leukocyte recruitment, including, e.g., neutrophil recruitment; leukocyte aggregation; leukocyte secretion of cytokines such as, e.g., IL-8; inflammation; adhesion; thrombosis; coagulation; and binding to a specific binding partner, e.g., P-selectin, L-selectin, or E-selectin. Futher, PSGL-1 activities associated with coagulation and thrombosis are included, such as modulating or inducing microparticle formation and recruitment, as well as tissue factor (TF) activity, i.e., circulating TF-microparticle activity. Downstream PSGL-1 activities include tyrosine phosphorylation, MAP kinase activation, and increased α_{M}b₂ binding activity. Clinical manifestations may include, e.g., redness, heat, increased temperature (which may be systemic or local), swelling, pain, loss of function, chills, fatigue/loss of energy, headache, loss of appetite, and muscle stiffness.

The term “PSGL-1 associated disorder,” as used herein, refers to a disease, disorder or condition associated with increased or aberrant PSGL-1 activity, expression, or localization. A PSGL-1 associated disorder includes a medical disorder such as a disorder associated with inflammation, thrombosis, coagulation, a T cell (i.e. CD8⁺) response, an immune disorder, or cardiovascular disorder, for example. PSGL-1 associated disorders include, but are not limited to, acute inflammatory diseases, adult respiratory distress syndrome, allergic conjunctivitis, allergies, such as a local or generalized allergic response, arterial injury, arthritis, asthma, atherosclerosis, autoimmune diseases, bacterial sepsis, bursitis, cancer, e.g., metastasis of tumor cells, circulatory shock, Crohn's disease, coagulopathy, colitis, coronary artery disease, coronary heart disease, deep vein thrombosis, disseminated intravascular coagulation, eczema, endotoxemic liver injury, gouty arthritis, graft versus host disease, hypercoagulability, irritable bowel disease, ileitis, inflammatory dermatosis, ischemia, leukaemia, multiple sclerosis, myocardial infarction, myocarditis, nasal polyposis, nephritis, organ transplant rejection, peritonitis, polymyalgia rheumatica, psoriasis, renal injury, renal ischemia, reperfusion injury, restenosis, rheumatoid arthritis, rhinitis, sepsis, sickle cell disease, solid organ transplantations stenosis, stroke, systemic inflammatory response syndrome, systemic lupus erythematosus, tendonitis, thrombocytopenia, including heparin-induced thrombocytopenia and thrombotic thrombocytopenic purpura, thrombosis, tumor metastasis, type I diabetes, ulcerative colitis, or venous thrombosis. Disorders of the heart, brain, lungs, kidneys, vascular system, and immune system are amenable to treatment with an antibody described herein.

The term “PSGL-1 inhibitor” includes any agent, such as, e.g., a neutralizing antibody, capable of inhibiting activity, expression, processing, or cell surface localization of PSGL-1. Such inhibitors are said to “inhibit,” “neutralize,” or “reduce” the biological activity of PSGL-1.

The term “reaction vessel” refers to a container in which an association of a molecule with an antibody that specifically binds to PSGL-1 can occur and be detected. A “surface” is the outer part of any solid such as, e.g., glass, cellulose, polyacrylamide, nylon, polystyrene, polyvinyl chloride, dextran sulfate, or treated polypropylene) to which an antibody can be directly or indirectly “contacted,” “immobilized,” or “coated.” A “surface of a reaction vessel” may be a part of the vessel itself, or the surface may be in the reaction vessel. A surface such as polystyrene, for example, may be subjected to chemical or radiation treatment to change the binding properties of its surface. Low binding, medium binding, high binding, aminated, and activated surfaces are encompassed by the term. An antibody can be directly contacted with a surface, e.g., by physical adsorption or a covalent bond to the surface, or it can be indirectly contacted, e.g., through an interaction with a substance or moiety that is directly contacted with the surface.

The term “repertoire” refers to a genetically diverse collection of nucleotide sequences derived wholly or partially from sequences encoding immunoglobulins. The sequences may be generated by rearrangement in vivo of the V, D, and J segments of heavy chains, and the V and J segments of light chains. Alternatively, the sequences can be generated from a cell in response to which rearrangement occurs, e.g., in vitro stimulation. Alternatively, part or all of the sequences may be obtained by DNA splicing, nucleotide synthesis, mutagenesis, and other methods (see, e.g., U.S. Pat. No. 5,565,332).

The term “specific interaction,” or “specifically binds,” or the like, means that two molecules form a complex that is relatively stable under physiologic conditions. The term is also applicable where, e.g., an antigen-binding domain is specific for a particular epitope, which is found on a number of molecules. Thus, an antibody may specifically bind multiple proteins when it binds to an epitope present in each. For example, an antibody described herein will bind to its antigen epitope in multiple contexts, such as, e.g., human PSGL-1 and their fragments, as well as fusion proteins comprising the same.

Specific binding is characterized by a selective interaction, often including high affinity binding with a low to moderate capacity. Nonspecific binding usually is a less selective interaction, and may have a low affinity with a moderate to high capacity. Typically, binding is considered specific when the affinity is at least 10⁶M⁻¹, or preferably at least 10⁷M⁻¹, or 10⁸M⁻¹, 10⁹M⁻¹, or 10¹⁰M⁻¹. If necessary, non-specific binding can be reduced without substantially affecting specific binding by varying the binding conditions. Such conditions are known in the art, and a skilled artisan using routine techniques can select appropriate conditions. The conditions are usually defined in terms of concentration of antibodies, ionic strength of the solution, temperature, time allowed for binding, concentration of non-related molecules (e.g., serum albumin, milk casein), etc. Exemplary conditions are set forth in the Examples.

Stringency, including “high stringency,” as used herein, includes conditions readily determined by the skilled artisan based on, for example, the length of the DNA. Generally, such conditions are defined as hybridization conditions of 50% formamide, 6×SSC at 42° C. (or other similar hybridization solution, such as, e.g., Stark's solution, in 50% formamide at 42° C.), and with washing at approximately 68° C., 0.2×SSC, 0.1% SDS. The skilled artisan will recognize that the temperature and wash solution salt concentration can be adjusted as necessary according to factors such as the length of the probe.

“Moderate stringency,” as used herein, includes conditions that can be readily determined by those having ordinary skill in the art based on, for example, the length of the DNA. The basic conditions are set forth by Sambrook et al., Molecular Cloning: A Laboratory Manual, 2d ed., 1:1.101-104, Cold Spring Harbor Laboratory Press (1989), and include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of 60° C., 0.5×SSC, 0.1% SDS.

The phrase “substantially as set out” means that the relevant CDR, V_{H}, or V_{L} domain will be either identical or highly similar to the specified regions of which the sequence is set out herein. For example, such substitutions include 1 or 2 substitutes, additions, or deletions for every approximately 5 amino acids in the sequence of a CDR (H1, H2, H3, L1, L2, or L3). A sequence is “substantially identical” if it has no more than 1 nucleic acid or amino acid residue substituted, deleted, or added for every 10-20 residues in the sequence.

The term “sulfated tyrosine” or “sulfotyrosine,” is used to include tyrosine-O-sulfate residues comprising a sulfate group covalently bound via the hydroxyl group of the tyrosine side chain. Alternatively, tyrosine may be O-sulfated at a terminal carboxyl group. An antibody may specifically bind to an epitope comprising one or more sulfotyrosine residues, but bind with much lower affinity to the epitope with one or more sulfated tyrosine residues. Sulfate may be added to a tyrosine by post-translational modification of a peptide or protein by incorporation of an optionally protected sulfotyrosine building block during peptide synthesis, by chemical synthesis, or by chemical alteration, for example. As used herein, “Y” indicates a tyrosine residue, while “y” indicates a sulfated tyrosine.

The term “treatment” is used interchangeably herein with the term “therapeutic method” and refers to both therapeutic treatment and prophylactic/preventative measures. Those in need of treatment may include individuals already having a particular medical disorder as well as those who may ultimately acquire the disorder (i.e., those needing preventative measures).

PSGL-1 Specific Antibodies

The present disclosure provides novel antibodies against primate PSGL-1 and antigen-binding fragments thereof. Nonlimiting illustrative embodiments of such antibodies are termed PSG3, PSG5, and PSG6. These exemplary embodiments are provided in the form of human IgG4 and/or IgG1 antibodies, and scFv fragments.

Antibodies described herein were selected for binding to a 19 amino acid fragment of human PSGL-1 comprising three sulfotyrosine residues. In certain embodiments, the antibodies specifically bind to the sulfated peptide, but not to the corresponding unsulfated peptide, which means that binding to the unsulfated form is not substantially above background levels. In one instance, a form of PSG3 antibody comprising a human IgG1 Fc with reduced Fc receptor binding and complement activation (PSG3-G1) (SEQ ID NO:37) increases the coagulation time of in vitro whole blood and plasma samples that were treated with a soluble P-selectin-Ig protein, completely inhibiting the P-selectin-dependent shortening of clotting time. Hrachovinova et al., Nature Med. 9:1020-1025 (2003). Further a PSG3 antibody blocks binding of platelets to leukocytes but does not induce leukocyte aggregation or stimulate the cells to release interleukin (IL-8). Id. at 1023.

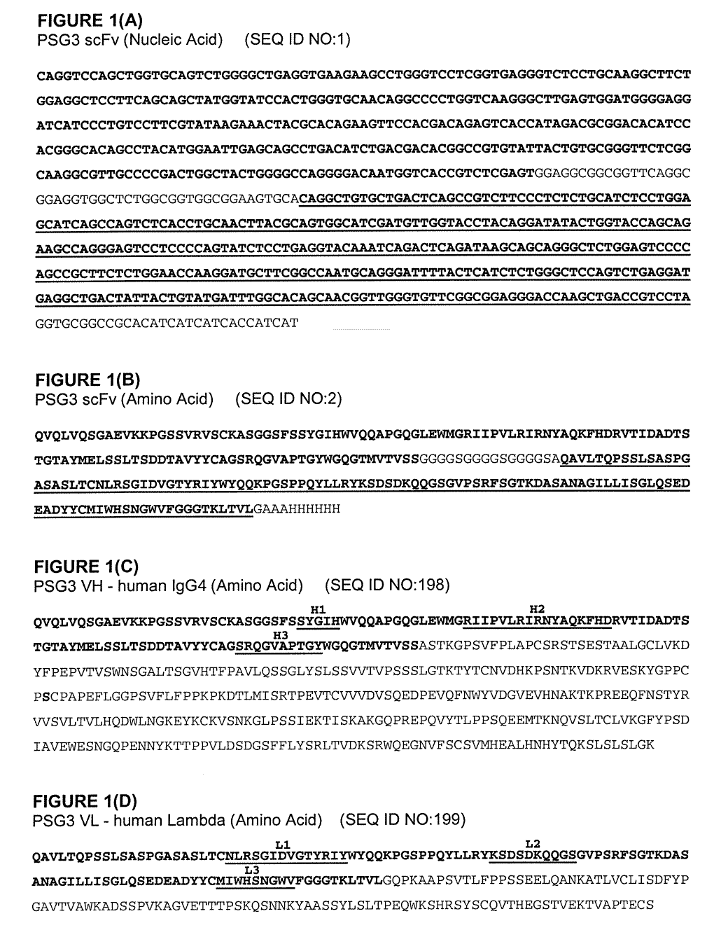

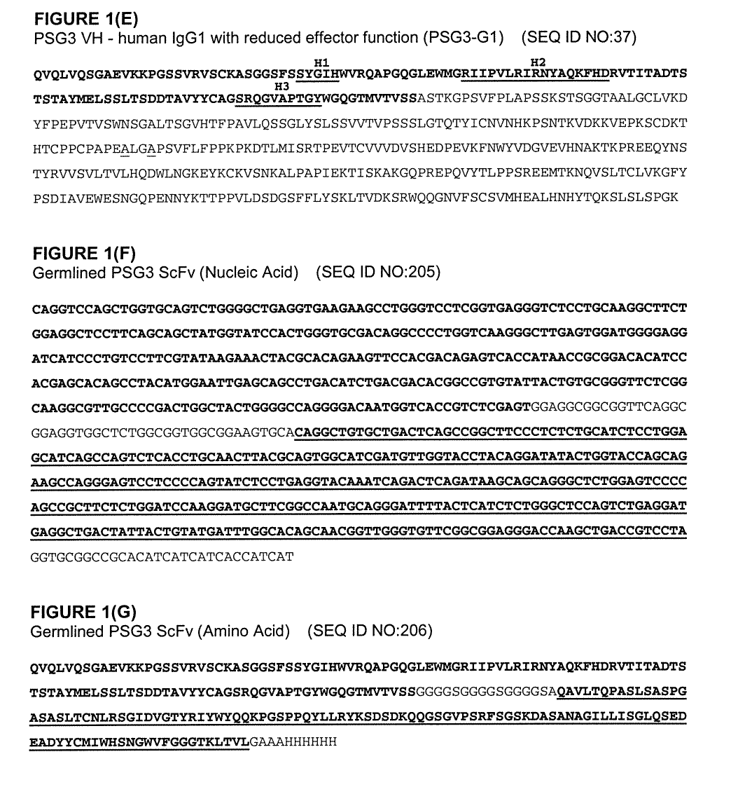

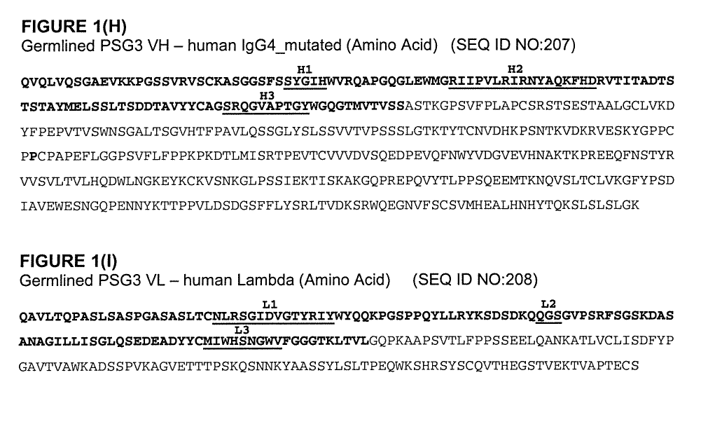

The antibodies of the invention are capable of specifically binding primate PSGL-1, and inhibiting one or more PSGL-1 activities in vitro and/or in vivo. Exemplary assays for evaluating PSGL-1 binding and/or inhibition of PSGL-1 activity include: assays measuring leukocyte adhesion (see, e.g., U.S. Patent Application Pub. No. 2004/0141966 A1, page 8, paragraphs 97-98); leukocyte rolling, e.g., by intravital microscopy (see, e.g., U.S. Patent Application Pub. No. 2003/0143662 A1, page 8, paragraphs 123-126); binding to P-selectin (see, e.g., U.S. Patent Publication Pub. No. 2002/0031508 A1, page 17, paragraph 165); P-selectin/PSGL-1 interaction (see, e.g., U.S. Patent Application Pub. No. 2005/0101569 A1, page 37, paragraphs 240-244; binding of a purified protein, including P-selectin or PSGL-1 to leukocytes, such as neutrophils (see, e.g., U.S. Patent Application Pub. No. 2003/0072755, page 5, paragraph 39); inflammation (see, e.g., U.S. Patent Application Pub. No. 2005/0141966 A1, page 8, paragraphs 99-101); tumor cell adhesion (see, e.g., U.S. Patent Application Pub. No. 2005/0181987A1, page 17, paragraph 140 and page 18, paragraph 147 to page 19, paragraph 153); platelet aggregation (see, e.g., U.S. Patent Application Pub. No. 2005/0004035 A1, column 8, paragraphs 80-81); coagulation (see, e.g., U.S. Patent Application Pub. No. 2002/0031508 A1, page 26, paragraph 264-275 and page 27, paragraphs 279-281; Hrachovinova et al., Nature Med. 9:1020-1025 (2003) (showing at page 1021 in FIG. 1 that PSG3 inhibits the pro-coagulant effect of soluble P-selectin-Ig fusion on whole blood or plasma in vitro); leukostasis (see, e.g., U.S. Patent Application Pub. No. 2004/0028648 A1, page 12, paragraphs 109-111); and PSGL-1 binding (see, e.g., U.S. Patent Application Pub. No. 2003/0166521 A1, page 18, paragraph 149 to page 19, paragraph 157), as well as the thrombosis and coagulation assays of and Cambien et al., Trends. Mol. Med. 10:179-186 (2004), for example.

One of ordinary skill in the art will recognize that antibodies of the invention may be used to detect, measure, and inhibit proteins that differ from those stated above.

In general, antibodies comprising an scFv or variable region set forth in SEQ ID NOs:2, 4, 6, 8, 10, 12, 14, 16, or 18 that specifically bind to SEQ ID NO:42 are provided herein. The disclosure also provides primate PSGL-1 specific antibodies that comprise at least one CDR of these antibodies (see, e.g., SEQ ID NOs:19 to 36).

Methods of making antibodies comprising SEQ ID NOs:2, 4, 6, 8, 10, 12, 14, 16, or 18 that specifically bind to human PSGL-1 or to an epitope comprising SEQ ID NO:42, SEQ ID NO:43, or SEQ ID NO:45 or are also provided. In one embodiment, these methods comprise transfecting a cell with a DNA construct, the construct comprising a DNA sequence encoding at least a portion of the neutralizing PSGL-1 specific antibodies of the invention, culturing the cell under conditions such that the antibody protein is expressed by the cell, and isolating the antibody protein.

In general, antibodies can be made, for example, using traditional hybridoma techniques (Kohler et al., Nature 256:495-499 (1975)), recombinant DNA methods (U.S. Pat. No. 4,816,567), or phage display performed with antibody libraries (Clackson et al., Nature 352:624-628 (1991); Marks et al., J. Mol. Biol. 222:581-597 (1991)). Antibodies are also produced recombinantly or synthetically. For other antibody production techniques, see also Antibodies: A Laboratory Manual, Harlow et al., Eds. Cold Spring Harbor Laboratory(1988) or Antibody Engineering, 2nd ed., Borrebaeck, Ed., Oxford University Press(1995) for example. Antibodies described herein are not limited to any particular source, species of origin, or method of production.

Intact antibodies, also known as immunoglobulins, are typically tetrameric glycosylated proteins composed of two light (L) chains of approximately 25 kDa each and two heavy (H) chains of approximately 50 kDa each. Two types of light chain, designated as the λ chain and the κ chain, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2.

The subunit structures and three-dimensional configurations of different classes of immunoglobulins are well known in the art. For a review of antibody structure, see Harlow et al., supra. Briefly, each light chain is composed of an N-terminal variable domain (VL) and a constant domain (CL). Each heavy chain is composed of an N-terminal variable domain (VH), three or four constant domains (CH), and a hinge region. The CH domain most proximal to VH is designated as CH1. The VH and VL domains consist of four regions of relatively conserved sequence called framework regions (FR1, FR2, FR3, and FR4), which form a scaffold for three regions of hypervariable sequence called complementarity determining regions (CDRs). The CDRs contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L 1, L2, and L3, accordingly. CDR3 and, particularly H3, are the greatest source of molecular diversity within the antigen-binding domain. H3, for example, can be as short as two amino acid residues or greater than 26.

The Fab fragment (Fragment antigen-binding) consists of the VH-CH1 and VL-CL domains covalently linked by a disulfide bond between the constant regions. To overcome the tendency of non-covalently linked VH and VL domains in the Fv to dissociate when co-expressed in a host cell, a so-called single chain (sc) Fv fragment (scFv) can be constructed. In an scFv, a flexible and adequately long linker connects either the C-terminus of the VH to the N-terminus of the VL or the C-terminus of the VL to the N-terminus of the VH. Most commonly, a 15-residue (Gly₄Ser)₃ peptide (SEQ ID NO:204) is used as a linker but other linkers are also known in the art.

The disclosure provides novel CDRs, and variable regions, derived from human immunoglobulin gene libraries. The structure for carrying a CDR, for example, will generally be an antibody heavy or light chain or a portion thereof, in which the CDR is located at a location corresponding to the CDR of naturally occurring V_{H} and V_{L}. The structures and locations of immunoglobulin variable domains may be determined, for example, as described in Kabat et al., Sequences of Proteins of Immunological Interest, No. 91-3242, National Institutes of Health Publications, Bethesda, Md.(1991).

DNA and amino acid sequences of PSGL-1 specific antibodies, their scFv fragments, VH and VL domains, and CDRs are set forth in the Sequence Listing and exemplary sequences are listed in Table 1. Particular nonlimiting illustrative embodiments of the antibodies are referred to as PSG3, PSG5, and PSG6. The CDR regions within the VH and VL domains of the illustrative embodiments are also listed in Table 1.

Table 1

DNA and Amino Acid (AA) Sequences of VH and VL
Domains and CDRs

| | |
|---|---|
| Sequence | PSG3 |

| | | | |
|---|---|---|---|
| scFv DNA | SEQ ID NO:1 | SEQ ID NO:7 | SEQ ID NO:13 |
| scFv AA | SEQ ID NO:2 | SEQ ID NO:8 | SEQ ID NO:14 |
| V_{H} DNA | SEQ ID NO:3 | SEQ ID NO:9 | SEQ ID NO:15 |
| V_{H} AA | SEQ ID NO:4 | SEQ ID NO:10 | SEQ ID NO:16 |
| V_{L} DNA | SEQ ID NO:5 | SEQ ID NO:11 | SEQ ID NO:17 |
| V_{L} AA | SEQ ID NO:6 | SEQ ID NO:12 | SEQ ID NO:18 |
| H₁ AA | SEQ ID NO:19 | SEQ ID NO:25 | SEQ ID NO:31 |
| H₂ AA | SEQ ID NO:20 | SEQ ID NO:26 | SEQ ID NO:32 |
| H₃ AA | SEQ ID NO:21 | SEQ ID NO:27 | SEQ ID NO:33 |
| L₁ AA | SEQ ID NO:22 | SEQ ID NO:28 | SEQ ID NO:34 |
| L₂ AA | SEQ ID NO:23 | SEQ ID NO:29 | SEQ ID NO:35 |
| L₃ AA | SEQ ID NO:24 | SEQ ID NO:30 | SEQ ID NO:36 |

PSGL-1 specific antibodies may optionally comprise antibody constant regions or parts thereof. For example, a V_{L} domain may have attached, at its C terminus, antibody light chain constant domains including human Ck or Cλ chains. Similarly, a specific antigen-binding domain based on a V_{H} domain may have attached all or part of an immunoglobulin heavy chain derived from any antibody isotope, e.g., IgG, IgA, IgE, and IgM and any of the isotope sub-classes, which include but are not limited to, IgG1 and IgG4. In the exemplary embodiments, PSG3, PSG5, and PSG6 antibodies comprise C-terminal fragments of heavy chains of IgG1 or IgG4 (see, e.g., Thompson et al., J. Immunol. Methods. 227:17-29 (1999)) and/or light chains of human IgG1λ, for example. The DNA and amino acid sequences for the C-terminal fragments are well known in the art (see, e.g., Kabat et al., Sequences of Proteins of Immunological Interest, No. 91-3242, National Institutes of Health Publications, Bethesda, Md.(1991); Thompson et al., J. Immunol. Methods 227:17-29 (1999)).

TABLE 2

| C-Terminal Region | Amino acid Sequence |
|---|---|
| IgG1 heavy chain | SEQ ID NO: 38 |
| IgG4 heavy chain | SEQ ID NO: 39 |
| λ light chain | SEQ ID NO: 40 |
| κ light chain | SEQ ID NO: 41 |

The portion of an immunoglobulin constant region can be a portion of an immunoglobulin constant region obtained from any mammal. The portion of an immunoglobulin constant region can include a portion of a human immunoglobulin, a non-human primate immunoglobulin, a bovine immunoglobulin, a porcine immunoglobulin, a murine immunoglobulin, an ovine immunoglobulin, or a rat immunoglobulin, for example.

The portion of an immunoglobulin constant region can include a portion of an IgG, an IgA, an IgM, an IgD, or an IgE. In one embodiment, the immunoglobulin is an IgG. In another embodiment, the immunoglobulin is an IgG₁. In yet another embodiment, the immunoglobulin is an IgG₄.

The portion of an immunoglobulin constant region can include the entire heavy chain constant region or a fragment or analog thereof. A heavy chain constant region can comprise a CH1 domain, a CH2 domain, a CH3 domain, and/or a hinge region, while a light chain constant region can comprise a CL domain. Thus, a constant region can comprise a CL, a CH1 domain, a CH2 domain, a CH3 domain, and/or a CH4 domain, for example.

The portion of an immunoglobulin constant region can include an Fc fragment. An Fc fragment can be comprised of the CH2 and CH3 domains of an immunoglobulin and the hinge region of the immunoglobulin. The Fc fragment can be the Fc fragment of an IgG1, an IgG2, an IgG3, or an IgG4. In one embodiment, the portion of an immunoglobulin constant region is an Fc fragment of an IgG1 or IgG4.

In some embodiments, the IgG constant region is modified to modulate (i.e. reduce or enhance) effector function as compared to the effector function of a wild-type immunoglobulin heavy chain Fc region. In various embodiments, the IgG constant region has reduced effector function, or alternatively it has increased effector function, for example. Fc effector function includes, for example, antibody-dependent cellular cytotoxicity (ADCC), phagocytosis, complement-dependent cytotoxicity, and half-life or clearance rate function. The IgG amino acid sequence of the Fc domain can be altered to affect binding to Fc gamma receptors (and thus ADCC or phagocytosis functions), to alter interaction with the complement system (complement-dependent cytotoxicity function), or with the neonatal Fc receptor (FcRn) (half-life), for example (see, e.g., Presta et al, Biochem. Society Transactions 30:487-490 (2002); U.S. Pat. No. 6,136,310). Methods of assaying T cell depleting activity, Fc effector function, and antibody half-life and pharmacokinetics are known in the art. In one embodiment, the antibody comprises a constant region or Fc portion that has low or no affinity for at least one Fc receptor. In an alternative embodiment, the second polypeptide has low or no affinity for complement protein C1q. In general, an effector function of an antibody can be altered by altering the affinity of the antibody for an effector molecule such as an Fc receptor. Binding affinity will generally be varied by modifying the effector molecule binding site. Disclosure of IgG modifications that alter interaction with effector molecules such as Fc receptors can be found in U.S. Pat. Nos. 5,624,821 and 5,648,260, in Presta, supra, as well as in references cited therein. For example, mutation of certain residues of IgG1 can reduce binding of IgG1 to all Fc receptors of the gamma subtype (e.g. Pro-238, Asp-265, Asp-270, Asn-297, or Pro-329 to alanine of human IgG1). Similarly, IgG Fc mutations that improve binding to FcRn are known, and can effect an increased half-life of the antibody in vivo. The residues of, for example, IgG1 that are important for interacting with Fc gamma receptors are generally distinct from those important for interacting with FcRn. Exemplary mutations for IgGs of various species are available. Combinations of mutations are included, and two or more mutations that increase binding affinity, for example, may be combined to yield a greater increase in binding affinity than either one alone.

In another embodiment, specific IgG1 heavy chain, IgG4 heavy chain, λ light chain, and κ light chain sequences are the basis for the immunoglobulin constant region. For example, in some embodiments the portion of an immunoglobulin constant region comprises SEQ ID NOs:38, 39, 40, or 41 or an analog fragment thereof. In another embodiment, the portion of an immunoglobulin constant region consists of SEQ ID NOs:38, 39, 40, or 41.

Certain embodiments comprise a V_{H} and/or V_{L} domain of an Fv fragment from PSG3, PSG5, or PSG6, i.e. SEQ ID NOs:4, 6, 10, 12, 16, or 18. Further embodiments comprise at least one CDR of any of these V_{H} and V_{L} domains. Antibodies comprising at least one of the CDR sequences of SEQ ID NOs:19-36 are encompassed within the scope of this invention. In one particular embodiment, for example, the antibodies comprise an H3 fragment of the V_{H} domain of PSG3, PSG5, or PSG6 (see, e.g., SEQ ID NOs:21, 27, and 33).

In certain embodiments, the V_{H} and/or V_{L} domains may be germlined. For example, the framework regions (FRs) of these domains are altered using molecular biology techniques to conform with those of the germline cells. A “germlined” sequence may be fully germlined or partially germlined, for example if some, but not all, variable domain residues conform with those of the germline cells. In other embodiments, the framework sequences remain diverged from the consensus germline sequences. In one embodiment, the germlined antibodies comprise at least one sequence of Table 1, for example.

In one embodiment, mutagenesis is used to make an antibody more similar to one or more germline sequences. This may be desirable when mutations are introduced into the framework region of an antibody through somatic mutagenesis in the individuals whose antibody V genes were used to construct a phagemid library, such as the library described in Example 1, or through error prone PCR used to increase variability in the CDRs in a library. Germline sequences for the V_{H} and V_{L} domains can be identified by performing amino acid and nucleic acid sequence alignments against the VBASE database (MRC Center for Protein Engineering, UK). VBASE is a comprehensive directory of all human germline variable region sequences compiled from over a thousand published sequences, including those in the current releases of the Genbank and EMBL data libraries. In some embodiments, the FR regions of the scFvs are mutated in conformity with the closest matches in the VBASE database and the CDR portions are kept intact.

In certain embodiments, the antibodies specifically bind an epitope comprising a PSGL-1 peptide in various amino acid sequence contexts. In some embodiments, the antibodies specifically bind to SEQ ID NO:42. For example, the antibodies may specifically bind to human PSGL-1 or its fragments that comprise sulfotyrosine, but not to human PSGL-1 or its fragments that comprise unsulfated tyrosine. In some embodiments, the antibody specifically binds its epitope with an affinity of at least 10⁷ M⁻¹, and preferably at least 10⁸ M⁻¹, 10⁹ M⁻¹, or 10¹⁰ M⁻¹.

It is contemplated that antibodies of the invention may also bind with high affinity to some PSGL-1 peptide sequences, and yet with low to moderate affinity to the same peptide sequences in some other three-dimensional contexts. Epitope mapping (see, e.g., Epitope Mapping Protocols, Morris, Ed., Humana Press (1996) and secondary and tertiary structure analyses can be carried out to identify specific 3D structures assumed by the disclosed antibodies and their complexes with antigens. Such methods include, but are not limited to, X-ray crystallography (Engstom, Biochem. Exp. Biol. 11:7-13 (1974)) and computer modeling of virtual representations of the presently disclosed antibodies (Fletterick et al., Computer Graphics and Molecular Modeling, in Current Communications in Molecular Biology, Cold Spring Harbor Laboratory,(1986)).

Derivatives

This disclosure also provides a method for obtaining an antibody that specifically binds to human PSGL-1. CDRs in such antibodies are not limited to the specific sequences of V_{H} and V_{L} identified in Table 1 and may include variants of these sequences that retain the ability to specifically bind sulfated tyrosine. Such variants may diverge from the sequences listed in Table 1, and be produced by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described in Antibody Engineering, 2^{nd} ed., Borrebaeck, Ed., Oxford University Press (1995). These include but are not limited to nucleotide sequences that are altered by the substitution of different codons that encode an identical or a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs (see Table 3). Furthermore, any native residue in the polypeptide may also be substituted with alanine (see, e.g., MacLennan et al., Acta Physiol. Scand. Suppl. 643:55-67 (1998); Sasaki et al., Adv. Biophys. 35:1-24 (1998)).

Conservative modifications will produce molecules having functional and chemical characteristics similar to those of the molecule from which such modifications are made. In contrast, substantial modifications in the functional and/or chemical characteristics of the molecules may be accomplished by selecting substitutions in the amino acid sequence that differ significantly in their effect on maintaining (1) the structure of the molecular backbone in the area of the substitution, for example, as a sheet or helical conformation, (2) the charge or hydrophobicity of the molecule at the target site, or (3) the size of the molecule.

For example, a “conservative amino acid substitution” may involve a substitution of a native amino acid residue with a nonnative residue such that there is little or no effect on the polarity or charge of the amino acid residue at that position. (See, for example, MacLennan et al., Acta Physiol. Scand. Suppl. 643:55-67 (1998); Sasaki et al., Adv. Biophys. 35:1-24 (1998)). Exemplary substitutions are set forth in Table 3.

Desired amino acid substitutions (whether conservative or non-conservative) can be determined by those skilled in the art at the time such substitutions are desired. For example, amino acid substitutions can be used to identify important residues of the molecule sequence, or to increase or decrease the affinity of the molecules described herein.

Derivatives and analogs of antibodies of the invention can be produced by various techniques well known in the art, including recombinant and synthetic methods (Sambrook et al., Molecular Cloning: A Laboratory Manual, 2^{nd} ed., Cold Spring Harbor Laboratory Press (1989), and Bodansky et al., The Practice of Peptide Synthesis, 2^{nd} ed., Spring Verlag, Berlin, Germany (1995)).

TABLE 3

| | | |
|---|---|---|
| Original | Exemplary | Typical |

| | | |
|---|---|---|
| Residues | Substitutions | Substitutions |

| | | |
|---|---|---|
| Ala (A) | Val, Leu, Ile, | Val |
| | 2-Aminobutanoic Acid | |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala, β-Alanine | Ala |
| His (H) | Asn, Gln, Lys, Arg | Arg |
| Ile (I) | Leu, Val, Met, Ala, Phe, | Leu |
| | Norleucine, Norvaline | |
| Leu (L) | Norleucine, Norvaline, Ile, | Ile |
| | Val, Met, Ala, Phe | |
| Lys (K) | Arg, Ornithine, | Arg |
| | 1,4-Diaminobutyric Acid, | |
| | 1,4-Diaminopropionic Acid, | |
| | Gln, Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, | Leu |
| | Norleucine, Norvaline | |

Antibodies provided herein also comprise a sequence that is at least about 70%, 80%, 85%, 90%, 92%, 93%, 94%, 95%, 96%, 97%, 98%, 99%, or more identical to any sequence of at least 100, 80, 60, 40, 20, 10, or 5 contiguous amino acids in the sequences as described herein. In general, proteins comprising an epitope for the antibodies provided herein may comprise a sequence that is at least about 70%, 80%, 85%, 90%, 92%, 93%, 94%, 95%, 96%, 97%, 98%, 99%, or more identical to any sequence of at least 18, 17, 16, 15, 14, 13, 12, 11, 10, 9, 8, 7, 6, 5, or 4contiguous amino acids in the sequence of human PSGL-1 set forth in SEQ ID NO:42. Nonlimiting examples of such proteins include sequences of PSGL-1 derived from various species. The percent identity is determined by standard alignment algorithms such as, for example, Basic Local Alignment Tool (BLAST) described in Altschul et al. J. Mol. Biol, 215:403-410 (1990), the algorithm of Needleman et al., J. Mol. Biol 48:444-453 (1970), or the algorithm of Meyers et al., Comput Appl. Biosci. 4:11-17 (1988).

In one embodiment, a method for making a V_{H} domain which is an amino acid sequence variant of a V_{H} domain of the invention comprises a step of adding, deleting, substituting, or inserting one or more amino acids in the amino acid sequence of the presently disclosed V_{H} domain, optionally testing the V_{H} domain thus provided with one or more V_{L} domains, or testing the V_{H} domain separately or in a different combination. Antibodies, including immunoglobulin fragments, are optionally tested for specific binding to primate PSGL-1 or a fragment thereof, for binding SEQ ID NO:42, or to a protein, such as, e.g., a fusion protein that comprises a PSGL-1 epitope, or for binding to a negative control such as a corresponding unsulfated PSGL-1 sequence. The ability of such antigen-binding domain to modulate the activity of human (or primate) PSGL-1, or another protein containing a PSGL-1 epitope, can also be tested. The V_{L} domain may have an amino acid sequence that is identical or is substantially as set out according to Table 1.

An analogous method can be employed in which one or more sequence variants of a V_{L} domain disclosed herein are combined with one or more V_{H} domains.

The antibodies described herein may be made by the procedures of Examples 1-2, and characterized by the assays of Examples 3-11, for example. A further aspect of the disclosure provides a method of preparing antigen-binding fragment that specifically binds with sulfated tyrosine. The method comprises:
a) providing a starting repertoire of nucleic acids encoding a V_{H} domain that either includes a CDR3 to be replaced or lacks a CDR3 encoding region;
(b) combining the repertoire with a donor nucleic acid encoding an amino acid sequence substantially as set out herein for a V_{H} CDR3 (i.e., H3) (see SEQ ID NOs:21, 27, or 33) such that the donor nucleic acid is inserted into the CDR3 region in the repertoire, so as to provide a product repertoire of nucleic acids encoding a V_{H} domain;
(c) expressing the nucleic acids of the product repertoire;
(d) selecting a binding fragment specific for PSGL-1 or a PSGL-1 epitope; and
(e) recovering the specific binding fragment or nucleic acid encoding it.

An analogous method may be employed in which a V_{L} CDR3 (i.e., L3) of the invention is combined with a repertoire of nucleic acids encoding a V_{L} domain, which either include a CDR3 to be replaced or lack a CDR3 encoding region. The donor nucleic acid may be selected from nucleic acids encoding an amino acid sequence substantially as set out in SEQ ID NOs:24, 30, or 36, for example.

A sequence encoding a CDR of the invention (e.g., CDR3) may be introduced into a repertoire of variable domains lacking the respective CDR (e.g., CDR3), using recombinant DNA technology, for example, using a methodology described by Marks et al., Bio/Technology 10:779-783 (1992). In particular, consensus primers directed at or adjacent to the 5′ end of the variable domain area can be used in conjunction with consensus primers to the third framework region of human V_{H} genes to provide a repertoire of V_{H} variable domains lacking a CDR3. The repertoire may be combined with a CDR3 of a particular antibody. Using analogous techniques, the CDR3-derived sequences may be shuffled with repertoires of V_{H} or V_{L} domains lacking a CDR3, and the shuffled complete V_{H} or V_{L} domains combined with a cognate V_{L} or V_{H} domain to make the sulfated tyrosine specific antibodies of the invention. The repertoire may then be displayed in a suitable host system such as the phage display system such as described in WO 92/01047 so that suitable antigen-binding fragments can be selected.

Analogous shuffling or combinatorial techniques are also disclosed by Stemmer, Nature 370:389-391 (1994), describing the technique in relation to a β-lactamase gene, but observing that the approach may be used for the generation of antibodies.

In further embodiments, one may generate novel V_{H} or V_{L} regions carrying one or more sequences derived from the sequences disclosed herein using random mutagenesis of one or more selected V_{H} and/or V_{L} genes. One such technique, error-prone PCR, is described in Gram et al., Proc. Natl. Acad. Sci. U.S.A. 89:3576-3580 (1992).

Another method that may be used is to direct mutagenesis to CDRs of V_{H} or V_{L} genes. Such techniques are disclosed in Barbas et al., Proc. Natl. Acad. Sci. U.S.A. 91:3809-3813 (1994) and Schier et al., J. Mol. Biol. 263:551-567 (1996).

Similarly, one or more, or all three, CDRs may be grafted into a repertoire of V_{H} or V_{L} domains, which are then screened for an antigen-binding fragment specific for sulfated tyrosine.

A portion of an immunoglobulin variable domain will comprise at least one of the CDRs substantially as set out herein and, optionally, intervening framework regions from the scFv fragments as set out herein. Residues at the N-terminal or C-terminal end of the variable domain may be heterologous, and may or may not be normally associated with naturally occurring variable domain regions. For example, construction of antibodies by recombinant DNA techniques may result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below. Secretion signals or affinity tags are examples of heterologous sequences of certain embodiments of the antibodies provided herein.

Although the embodiments illustrated in the Examples comprise a “matching” pair of V_{H} and V_{L} domains, a skilled artisan will recognize that alternative embodiments may comprise antigen-binding fragments containing only a single CDR from either V_{L} or V_{H} domain or any combination of CDR sequences. Either of the single chain specific binding domains can be used to screen for complementary domains capable of forming a two-domain specific antigen-binding fragment capable of, for example, binding to sulfated tyrosine. The screening may be accomplished by phage display screening methods using the so-called hierarchical dual combinatorial approach disclosed in WO 92/01047, for example, in which an individual colony containing either an H or L chain clone is used to infect a complete library of clones encoding the other chain (L or H), and the resulting two-chain specific binding domain is selected in accordance with phage display techniques as described.

The PSGL-1 specific antibodies described herein can be linked to another functional and/or stabilizing molecule. For example, antibodies may be linked to another peptide or protein (albumin, another antibody, etc.), toxin, radioisotope, cytotoxic or cytostatic agents. The antibodies can be linked covalently by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The antibodies can be chemically modified by covalent conjugation to a polymer, for example, to increase their stability or half-life. Exemplary polymers and methods to attach them are also shown in U.S. Pat. Nos. 4,766,106; 4,179,337; 4,495,285; and 4,609,546.

The disclosed antibodies may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain one or more glycosylation site consensus sequences known in the art. Another means of increasing the number of carbohydrate moieties on the antibodies is by chemical enzymatic coupling of glycosides to the amino acid residues of the antibody. Such methods are described in WO 87/05330 and in Aplin et al., CRC Crit. Rev. Biochem. 22:259-306 (1981). Removal of any carbohydrate moieties from the antibodies may be accomplished chemically or enzymatically, for example, as described by Hakimuddin et al., Arch. Biochem. Biophys. 259:52 (1987); and Edge et al., Anal. Biochem. 118:131 (1981), and by Thotakura et al., Meth. Enzymol. 138:350 (1987).

The antibodies may also be tagged with a detectable label. A detectable label is a molecule which, by its chemical nature, provides an analytically identifiable signal which allows the detection of a molecular interaction. A protein, including an antibody, has a detectable label if it is covalently or non-covalently bound to a molecule that can be detected directly (e.g., by means of a chromophore, fluorophore, or radioisotope) or indirectly (e.g., by means of catalyzing a reaction producing a colored, luminescent, or fluorescent product). Detectable labels include a radiolabel such as ¹³¹I or ⁹⁹Tc, a heavy metal, or a fluorescent substrate, such as Europium, for example, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

Antibodies in which CDR sequences differ only insubstantially from those of the variable regions of PSG3, PSG5, and PSG6 are encompassed within the scope of this invention. Typically, an amino acid is substituted by a related amino acid having similar charge, hydrophobic, or stereochemical characteristics. Such substitutions would be within the ordinary skills of an artisan. A skilled artisan would appreciate that changes can be made in FRs without adversely affecting the binding properties of an antibody. Changes to FRs include, but are not limited to, humanizing a non-human derived or engineering certain framework residues that are important for antigen contact or for stabilizing the binding site, e.g., changing the class or subclass of the constant region, changing specific amino acid residues which might alter the effector function such as Fc receptor binding, e.g., as described in U.S. Pat. Nos. 5,624,821 and 5,648,260 and Lund et al., J. Immunol. 147:2657-2662 1991) and Morgan et al., Immunology 86:319-324 (1995), or changing the species from which the constant region is derived.

One of skill in the art will appreciate that the modifications described above are representative only, and that many other modifications would be obvious to a skilled artisan in light of the teachings of the present disclosure.

Nucleic Acids, Cloning, and Expression Systems

The present disclosure further provides isolated nucleic acids encoding the disclosed antibodies. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

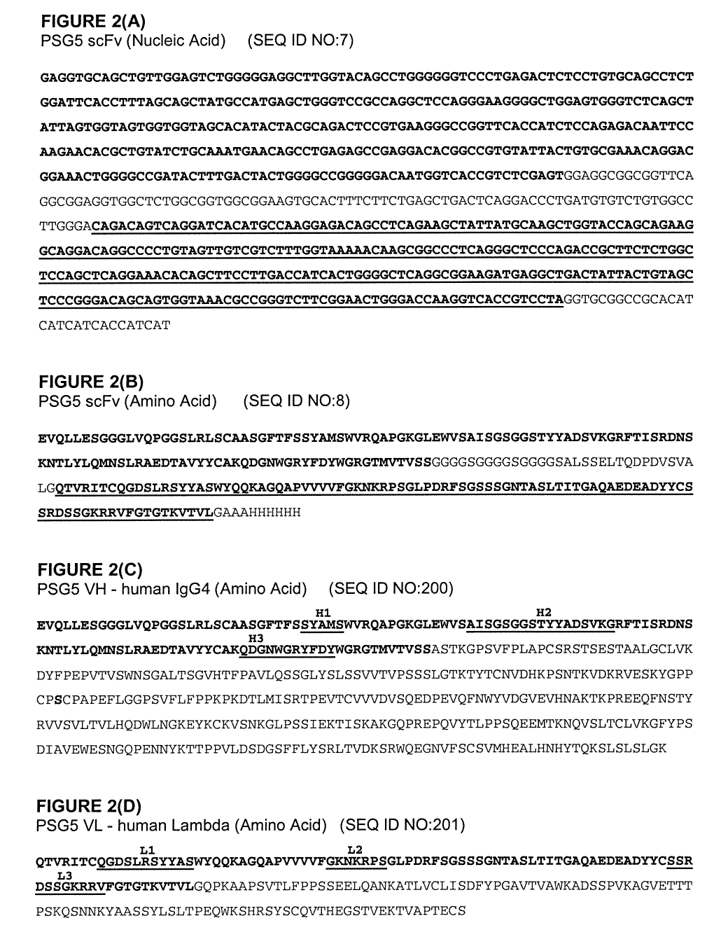

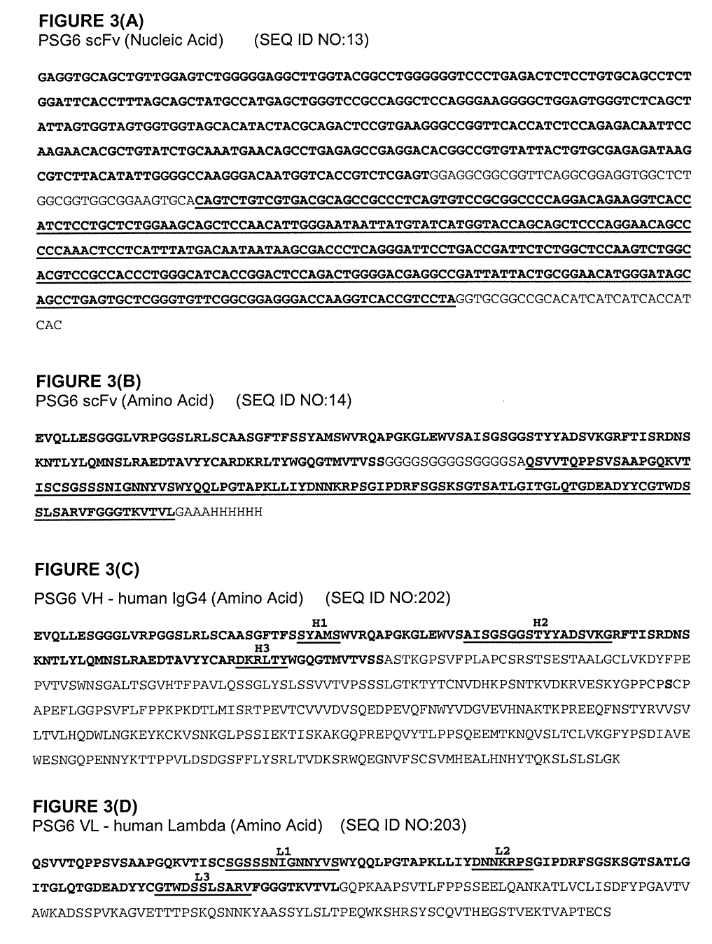

The nucleic acids provided herein comprise a coding sequence for a CDR, a V_{H} domain, and/or a V_{L} domain disclosed herein. Similarly, nucleic acid fragments encoding portions of these antibodies are disclosed. In one embodiment, the nucleic acid construct comprises the DNA sequence of FIG. 1(A) (SEQ ID NO:1) or a homolog thereof. In another embodiment, the nucleic acid construct comprises the DNA sequence of FIG. 2(A) (SEQ ID NO:7) or an analog thereof, or the DNA sequence of FIG. 3(A) (SEQ ID NO:13) or an analog thereof. The DNA optionally comprises, e.g., SEQ ID NOs:3, 5, 9, 11, 15, or 17. In another embodiment, the nucleic acid construct comprises a nucleic acid that encodes one or more antibody sequences set forth in the sequence listing.

The present disclosure also provides constructs in the form of plasmids, vectors, phagemids, transcription or expression cassettes which comprise at least one nucleic acid encoding a CDR, a V_{H} domain, and/or a V_{L} domain disclosed herein.

The disclosure further provides a host cell which comprises one or more constructs as above.

Also provided are nucleic acids encoding any CDR(H1, H2, H3, L1, L2, or L3), V_{H} or V_{L} domain, as well as methods of making the encoded products. The method comprises expressing the encoded product from the encoding nucleic acid. Production may be achieved by culturing recombinant host cells containing the nucleic acid under appropriate conditions. Following production, a V_{H} or V_{L} domain or other antibody or specific fragment may be isolated and/or purified using any suitable technique, then used as appropriate.

Antigen-binding fragments, V_{H} and/or V_{L} domains, and the nucleic acid molecules and vectors encoding the same may be isolated and/or purified from their natural environment, in substantially pure or homogeneous form, or, in the case of nucleic acid, free or substantially free of nucleic acid or other contaminating factors.

The invention also provides isolated DNA sequences encoding polypeptides of the invention that differ from a reference antibody sequence, but retain the antigen specificity. For example, variant sequences are provided which encode a polypeptide that specifically binds to PSGL-1 or a fragment thereof, but not to the corresponding unsulfated polypeptide. Due to the known degeneracy of the genetic code, wherein more than one codon can encode the same amino acid, a DNA sequence can vary from that shown in SEQ ID NOs:1, 3, 5, 7, 9, 11, 13, 15, or 17 and still encode a polypeptide having the amino acid sequence of SEQ ID NOs:2, 4, 6, 8, 10, 12, 14, 16, or 18, for example. Such variant DNA sequences can result from naturally occurring, accidental, and/or deliberate mutagenesis of a native sequence. A nucleic acid capable of hybridizing to a nucleic acid that encodes a human PSGL-1 specific antibody under high stringency conditions is also described herein.

In another embodiment, the nucleic acid molecules of the invention also comprise nucleotide sequences that are at least 80% identical or that encode an amino acid that is at least 80% identical to a native sequence. Also contemplated are embodiments in which a sequence is at least 85%, 90%, 95%, 96%, 97%, 98%, 99%, or 99.5% identical to a reference sequence. The percent identity may be determined by visual inspection and mathematical calculation. Alternatively, the percent identity of two nucleic acid sequences can be determined by comparing sequence information using the GAP computer program, version 6.0 described by Devereux et al., Nucl. Acids Res. 12:387 (1984) and available from the University of Wisconsin Genetics Computer Group (UWGCG).

Systems for cloning and expression of a polypeptide in a variety of different host cells are well known in the art. For cells suitable for producing antibodies, see Gene Expression Systems; Fernandez et al., Eds.; Academic Press, 1999. Briefly, suitable host cells include bacteria, yeast, insect, plant, animal, and mammalian cells, and yeast and baculovirus expression systems may be appropriate. Mammalian cell lines available in the art for expression of a heterologous polypeptide include Chinese Hamster Ovary cells, HeLa cells, baby hamster kidney cells, NS0 mouse myeloma cells, and many others. A common bacterial host is E. coli. Any protein expression system compatible with the invention may be used to produce the disclosed antibodies. Suitable expression systems include transgenic animals described in Gene Expression Systems; Fernandez et al., Eds.; Academic Press, 1999.

Suitable vectors or DNA constructs can be chosen or constructed, so that they contain appropriate regulatory sequences, including promoter sequences, terminator sequences, polyadenylation sequences, enhancer sequences, marker or selection genes, and other sequences as appropriate. Constructs may be plasmids or viral, e.g., phage, or phagemid, as appropriate. In one embodiment, the nucleic acid construct is comprised of DNA. In another embodiment, the nucleic acid construct is comprised of RNA. The nucleic acid construct can be a vector, e.g., a viral vector or a plasmid. Examples of viral vectors include, but are not limited to, adeno virus vector, an adeno-associated virus vector, or a murine leukemia virus vector. Examples of plasmids include, but are not limited to, pUC and pGEX. For further details see, for example, Sambrook et al., Molecular Cloning: A Laboratory Manual, 2^{nd} ed., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2^{nd} ed., Ausubel et al., Eds., John Wiley & Sons, 1992.

A further aspect of the disclosure provides a host cell comprising a nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage, for example. The introduction of the nucleic acid into the cells may be followed by causing or allowing expression from the nucleic acid, e.g., by culturing host cells under conditions for expression of the gene.

Production of Antibody Proteins

Antibody proteins of the invention can be produced using techniques well known in the art. For example, the antibody proteins of the invention can be produced recombinantly in cells (see, e.g., Sambrook et al., Molecular Cloning: A Laboratory Manual, Cold Spring Harbor Laboratory, N.Y., 1989, and Ausubel et al., Current Protocols in Molecular Biology, Greene Publishing Associates and Wiley Interscience, N.Y., 1989). Alternatively, the antibody proteins of the invention can be produced using known synthetic methods such as solid phase synthesis. Synthetic techniques are well known in the art (see, e.g., Merrifield, Chemical Polypeptides, Katsoyannis and Panayotis Eds., 1973, pp. 335-61; Merrifield, J. Am. Chem. Soc. 85:2149 (1963); Davis et al., Biochem. Intl. 10:394 (1985); Finn et al., The Proteins (3^{rd} ed.) 2:105 (1976); Erikson et al., The Proteins (2^{nd} ed.) 2:257 (1976); U.S. Pat. No. 3,941,763). Further, the antibody proteins of the invention can be produced using a combination of recombinant and synthetic methods. In certain applications, it may be beneficial to use either a recombinant method or a combination of recombinant and synthetic methods.

For recombinant production, a polynucleotide sequence encoding the antibody protein is inserted into an appropriate expression vehicle, such as a vector which contains the necessary elements for the transcription and translation of the inserted coding sequence, or in the case of an RNA viral vector, the necessary elements for replication and translation. The nucleic acid encoding the antibody protein is inserted into the vector in proper reading frame.

The expression vehicle is then transfected into a suitable target cell which will express the peptide. Transfection techniques known in the art include, but are not limited to, calcium phosphate precipitation (Wigler et al., Cell 14:725 (1978)) and electroporation (Neumann et al., EMBO J. 1:841 (1982)). A variety of host-expression vector systems may be utilized to express the antibody proteins described herein including both prokaryotic (e.g., E. coli) or eukaryotic cells. These include, but are not limited to, microorganisms such as bacteria (e.g., E. coli) transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems, including mammalian cells (e.g., CHO cells, Cos cells, HeLa cells, myeloma cells).

When the antibody protein is expressed in a eukaryotic cell, the DNA encoding the antibody protein may also code for a signal sequence that will permit the antibody protein to be secreted. One skilled in the art will understand that a signal sequence is translated and that it may be cleaved from the polypeptide to form the mature antibody protein. Various signal sequences are known in the art, e.g., the interferon a signal sequence and the mouse Igk light chain signal sequence. Alternatively, where a signal sequence is not included the antibody protein can be recovered by lysing the cells.

When the antibody protein of the invention is recombinantly synthesized in a prokaryotic cell, it maybe desirable to refold the protein. The antibody protein produced by this method can be refolded to a biologically active conformation using conditions known in the art, e.g., denaturing and reducing conditions and then slow dialysis in PBS.

Depending on the expression system used, the expressed peptide is then isolated by procedures well-established in the art (e.g., affinity chromatography, size exclusion chromatography, and/or ion exchange chromatography).

The expression vectors can encode an affinity tag to permit easy purification of the recombinantly produced protein. Examples include, but are not limited to, histidine tags, flag tags, and maltose protein binding tags. For example, vector pUR278 (Ruther et al., EMBO J. 2:1791 (1983)) may be used in which the coding sequence of the antibody of the invention may be ligated into the vector in frame with the lac z coding region so that a hybrid protein is produced. In another example, pGEX vectors may be used to express proteins with a glutathione S-transferase (GST) tag. GST fusion proteins are often soluble and can be purified from cells by adsorption to glutathione-agarose beads followed by elution in the presence of free glutathione. The vectors optimally include cleavage sites (thrombin or factor Xa protease or PreScission Protease™ (Pharmacia, Peapack, N.J.)) for removal or cleavage of the tag after purification of the polypeptide.

Vectors used in transformation will usually contain a selectable marker used to identify transformants. In bacterial systems this can include an antibiotic resistance gene such as ampicillin or kanamycin. Selectable markers for use in cultured mammalian cells include genes that confer resistance to drugs, such as neomycin, hygromycin, and methotrexate. The selectable marker may be an amplifiable selectable marker. One amplifiable selectable marker is the DHFR gene. Another amplifiable marker is the DHFR cDNA (Simonsen and Levinson, Proc. Natl. Acad. Sci. U.S.A. 80:2495 (1983)). Selectable markers are reviewed by Thilly (Mammalian Cell Technology, Butterworth Publishers, Stoneham, Mass.) and the choice of selectable markers is well within the level of ordinary skill in the art.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used. When cloning in insect cell systems, promoters such as the baculovirus polyhedron promoter may be used. When cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used. When cloning in mammalian cell systems, promoters derived from the genome of mammalian cells (e.g., metallothionein promoter) or from mammalian viruses (e.g., the adenovirus late promoter; the vaccinia virus 7.5 K promoter; the CMV promoter) may be used; when generating cell lines that contain multiple copies of expression product, SV40-, BPV- and EBV-based vectors may be used with an appropriate selectable marker.

In cases where plant expression vectors are used, the expression of sequences encoding linear or non-cyclized forms of the antibody proteins of the invention may be driven by any of a number of promoters. For example, viral promoters such as the 35S RNA and 19S RNA promoters of CaMV (Brisson et al., Nature 310:511-514 (1984)), or the coat protein promoter of TMV (Takamatsu et al., EMBO J. 6:307-311 (1987)) may be used; alternatively, plant promoters such as the small subunit of RUBISCO (Coruzzi et al., EMBO J. 3:1671-1680 1984); Broglie et al., Science 224:838-843 (1984)) or heat shock promoters, e.g., soybean hsp17.5-E or hsp 17.3-B (Gurley et al., Mol. Cell. Biol. 6:559-565 (1986)) may be used. These constructs can be introduced into plant cells using Ti plasmids, Ri plasmids, plant virus vectors, direct DNA transformation, microinjection, electroporation, etc. For reviews of such techniques see, e.g., Weissbach & Weissbach, Methods for Plant Molecular Biology, Academic Press, NY, Section VIII, pp. 421-463 (1988); and Grierson & Corey, Plant Molecular Biology, 2d ed., Blackie, London, Ch. 7-9 (1988).

In one insect expression system that may be used to produce the antibody proteins of the invention, Autographa californica nuclear polyhidrosis virus (AcNPV) is used as a vector to express the foreign genes. The virus grows in Spodoptera frugiperda cells. A coding sequence for a heterologous polypeptide may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al., J. Virol. 46:584 (1983); U.S. Pat. No. 4,215,051). Further examples of this expression system may be found in Ausubel et al., Eds, Current Protocols in Molecular Biology, Vol.2, Greene Publish. Assoc. & Wiley Interscience (1989).

In mammalian host cells, a number of expression systems may be utilized, such as viral-based systems. In cases where an adenovirus is used as an expression vector, a coding sequence may be ligated to an adenovirus transcription/translation control complex, e.g., the late promoter and tripartite leader sequence. This antibody gene may then be inserted in the adenovirus genome by in vitro or in vivo recombination.

In cases where an adenovirus is used as an expression vector, a coding sequence may be ligated to an adenovirus transcription/translation control complex, e.g., the late promoter and tripartite leader sequence. This antibody gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts (see, e.g., Logan & Shenk, Proc. Natl. Acad. Sci. U.S.A. 81:3655-3659 (1984)). Alternatively, the vaccinia 7.5 K promoter may be used (see, e.g., Mackett et al., Proc. Natl. Acad. Sci. U.S.A. 79:7415-7419 (1982); Mackett et al., J. Virol. 49:857-864 (1984); Panicali et al., Proc. Natl. Acad. Sci. U.S.A. 79:4927(1982)).

Host cells containing DNA constructs of the antibody protein are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals, and growth factors. Optionally, the media can contain bovine calf serum or fetal calf serum. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum containing or serum-free media (e.g., MEM, DMEM). Selection of a medium appropriate for the particular cell line used is within the level of ordinary skill in the art.

The recombinantly produced antibody protein of the invention can be isolated from culture media. The culture medium from appropriately grown transformed or transfected host cells is separated from the cell material, and the presence of antibody proteins is demonstrated. One method of detecting the antibody proteins, for example, is by the binding of the antibody proteins or portions of the antibody proteins to a specific antibody recognizing the antibody protein of the invention (e.g., an anti-Fc antibody). An anti-antibody protein antibody may be a monoclonal or polyclonal antibody raised against the antibody protein in question. For example, the antibody protein can contain a portion of an immunoglobulin constant region. Antibodies recognizing the constant region of many immunoglobulins are known in the art and are commercially available. An antibody can be used to perform an ELISA or a western blot to detect the presence of the antibody protein of the invention.

The antibody protein of the invention can be produced in a transgenic animal, such as a rodent. The term “transgenic animals” refers to non-human animals that have incorporated a foreign gene into their genome. Because this gene is present in germline tissues, it is passed from parent to offspring. Methods of producing transgenic animals are known in the art, including transgenics that produce immunoglobulin molecules (Wagner et al., Proc. Natl. Acad. Sci. U.S.A. 78:6376 (1981); McKnight et al., Cell 34:335 (1983); Brinster et al., Nature 306:332 (1983); Ritchie et al., Nature 312:517(1984)).

The invention also relates to a pharmaceutical composition comprising one or more PSGL specific antibodies or active portions thereof and a pharmaceutically acceptable carrier or excipient. The compositions may also contain other active compounds providing supplemental, additional, or enhanced therapeutic functions. Examples of suitable pharmaceutical carriers are described in Remington's Pharmaceutical Sciences by E. W. Martin. Examples of excipients can include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like as well as those described infra. The composition optionally contains pH buffering reagents and wetting or emulsifying agents. The pharmaceutical compositions may also be included in a container, pack, or dispenser together with instructions for administration.

The presently disclosed antibodies may be prepared with carriers that will protect the compound against rapid elimination from the body, such as a controlled release formulation, including implants and microencapsulated delivery systems.

Methods to Treat PSGL-1 Associated Disorders

The antibodies of the present invention are useful to prevent, diagnose, and/or treat various medical disorders in humans or animals. The antibodies can be used to inhibit or reduce one or more activities associated with PSGL-1, or associated with a related protein. For example, the antibodies may inhibit or reduce one or more of the activities of PSGL-1 relative to the PSGL-1 that is not bound by an antibody. In certain embodiments, the antibodies inhibit the activity of PSGL-1 at least 50%, 55%, 60%, 65%, 70%, 75%, 80%, 85%, 90%, 95%, 96%, 97%, 98%, or 99%. Inhibition of PSGL-1 activity can be measured by a number of in vivo and in vitro assays, as discussed infra.

PSGL-1 has functional importance in leukocyte platelet, and/or microvesicle adhesion, rolling, recruitment, aggregation; leukocyte secretion of cytokines; promotion of coagulation; and other aspects of inflammation, thrombosis, coagulation, immune response, and signal transduction. PSGL-1 is also involved in tumor metastasis. A neutralizing antibody described herein will inhibit one or more of these PSGL-1 activities, in vivo or in vitro, for example. Thus, the inhibition of PSGL-1 with a neutralizing antibody described herein is useful in the treatment of various disorders associated with inflammation, thrombosis, coagulation, T cell response, as well as in the treatment of immune and cardiovascular disorders, for example.

In another aspect, the PSGL-1 specific antibodies disclosed herein may bind preferentially to an epitope expressed, or expressed at a higher level, on a diseased cell. In such embodiments, the antibodies can induce antibody-dependent cytotoxicity and/or can stimulate natural killer (NK) or T cells.

The medical disorder being diagnosed, treated, or prevented by the presently disclosed antibodies is a PSGL-1 associated disorder, such as, e.g., a disorder related to leukocyte rolling, leukocyte adhesion, leukocyte migration, microvesicle formation and/or recruitment, thrombosis, coagulation, immune response, tumor metastasis, or inflammation.

The invention relates to a method of treating a subject having or at risk for developing a disorder in which one or more symptoms or manifestations of the disorder are improved by modulating the activity of PSGL-1. The antibody proteins of the invention can be used to treat or prevent disorders that result from P-selectin L-selectin, and/or E-selectin binding. In particular embodiments, the antibodies of the invention are used to treat or prevent disorders such as, e.g., acute inflammatory diseases, adult respiratory distress syndrome, allergic conjunctivitis (such as a local or generalized allergic response), arterial injury, allergies, arthritis, asthma, atherosclerosis, autoimmune diseases, bacterial sepsis, bursitis, cancer, e.g., metastasis of tumor cells, circulatory shock, Crohn's disease, coagulopathy, colitis, coronary artery disease, coronary heart disease, deep vein thrombosis, disseminated intravascular coagulation, eczema, endotoxemic liver injury, gouty arthritis, hypercoagulability, irritable bowel disease, graft versus host disease, type I diabetes, ileitis, inflammatory dermatosis, ischemia, leukaemia, multiple sclerosis, myocardial infarction, myocarditis, nasal polyposis, nephritis, organ transplant rejection, peritonitis, polymyalgia rheumatica, psoriasis, renal injury, renal ischemia, reperfusion injury, restenosis, rheumatoid arthritis, rhinitis, sepsis, sickle cell disease, solid organ transplantation, stenosis, stroke, systemic inflammatory response syndrome, systemic lupus erythematosus, tendonitis, thrombocytopenia, including heparin-induced thrombocytopenia and thrombotic thrombocytopenic purpura, thrombosis, tumor metastasis, ulcerative colitis, and venous thrombosis. Disorders of the heart, brain, lungs, kidneys, vascular system, and immune system are amenable to treatment with an antibody described herein.

Cardiovascular diseases and disorders include arteriosclerosis, ischemia/reperfusion injury, arterial inflammation, rapid ventricular pacing, coronary microembolism, tachycardia, bradycardia, pressure overload, aortic bending, vascular heart disease, atrial fibrillation, Jervell syndrome, Lange-Nielsen syndrome, Long QT syndrome, congestive heart failure, sinus node dysfunction, angina, heart failure, hypertension, atrial fibrillation, atrial flutter, cardiomyopathy, e.g., dilated cardiomyopathy and idiopathic cardiomyopathy, myocardial infarction, coronary artery disease, coronary artery spasm, and arrhythmia, for example.

In certain embodiments, the immune response of an individual is reduced at least 10%, 20%, 30%, 40%, 50%, 55%, 60%, 65%, 70%, 75%, or 80% upon administration of one or more of the presently disclosed antibodies, as measured by, for example, levels of TNF-α, leukocyte-produced oxidants, procalcitonin, leukocyte high-affinity Fc receptor (CD64), serum C-reactive protein, high mobility group protein 1, IL-1 (e.g., IL-1β), IL-6, IL-8, or platelet activating factor (PAF). In other embodiments, administration of one or more of the presently disclosed antibodies results in a decrease in bacterial or bacterial endotoxin levels.

The antibodies or antibody compositions of the present invention are administered in therapeutically effective amounts. Generally, a therapeutically effective amount may vary with the subject's age, condition, and sex, as well as the severity of the medical condition in the subject. The dosage may be determined by a physician and adjusted, as necessary, to suit observed effects of the treatment. Toxicity and therapeutic efficacy of such compounds can be determined by standard pharmaceutical procedures in vitro (i.e., cell cultures) or in vivo (i.e., experimental animal models), e.g., for determining the LD₅₀ (the dose lethal to 50% of the population) and the ED₅₀ (the dose therapeutically effective in 50% of the population). The dose ratio between toxic and therapeutic effects is the therapeutic index (or therapeutic ratio), and can be expressed as the ratio LD₅₀/ED₅₀. Antibodies that exhibit therapeutic indices of at least 1, 1.5, 2, 3, 4, 5, 6, 7, 8, 9, 10, and 20 are described herein. Antibodies that exhibit a large, therapeutic index are preferred.

The data obtained from in vitro assays and animal studies, for example, can be used in formulating a range of dosage for use in humans. The dosage of such compounds lies preferably within a range of circulating concentrations that include the ED₅₀ with low, little, or no toxicity. The dosage may vary within this range depending upon the dosage form employed and the route of administration utilized. For any antibody used in the present invention, the therapeutically effective dose can be estimated initially from in vitro assays. A dose may be formulated in animal models to achieve a circulating plasma concentration range that includes the IC₅₀ (i.e., the concentration of the test antibody which achieves a half-maximal inhibition of symptoms) as determined in in vitro experiments. Levels in plasma may be measured, for example, by high performance liquid chromatography. The effects of any particular dosage can be monitored by a suitable bioassay, such as a coagulation assay.

Generally, the compositions are administered so that antibodies or their binding fragments are given at a dose between 1 μg/kg and 30 mg/kg, 1 μg/kg and 10 mg/kg, 1 μg/kg and 1 mg/kg, 10 μg/kg and 1 mg/kg, 10 μg/kg and 100 μg/kg, 100 μg and 1 mg/kg, and 500 μg/kg and 1 mg/kg. In some embodiments, the antibodies are given as a bolus dose, to maximize the circulating levels of antibodies for the greatest length of time after the dose. Continuous infusion may also be used after the bolus dose.

Optionally, the primate PSGL-1 specific antibody is administered in combination with a second therapeutic agent. Exemplary second therapeutic agents include anti-coagulant or anti-thrombotic agents, e.g. heparin (including low molecular weight heparin) and tissue factor plasminogen activator (TPA) (see Example 9). The second therapeutic agent may be, for example, an anti-cancer agent, anti-neoplastic agent, anti-viral agent (e.g., acyclovir, ganciclovir or zidovudine), anti-metastatic agent, anti-inflammatory agent (e.g., zaltoprofen, pranoprofen, droxicam, acetyl salicylic 17, diclofenac, ibuprofen, dexibuprofen, sulindac, naproxen, amtolmetin, celecoxib, indomethacin, rofecoxib, or nimesulid), anti-thrombosis agent (e.g., cilostazol, dalteparin sodium, reviparin sodium, or aspirin), anti-restenosis agent, anti-aggregation agent, anti-autoimmune agent e.g., leflunomide, denileukin diftitox, subreum, WinRho SDF, defibrotide, orcyclophosphamide), anti-adhesion agent (e.g., limaprost, clorcromene, or hyaluronic acid), anti-cardiovascular disease agent, pharmaceutical agent, or other therapeutic agent. In some embodiments, the PSGL-1 specific antibodies are administered with one or more of dopamine, norepinephrine, mannitol, furosemide, digitalis, pyridoxylated hemoglobin polyoxyethylene, prostaglandin E1, granulocyte colony stimulation factor (GCSF), and antibodies to various antigens on bacterial cell walls or to bacterial endotoxin. These second therapeutic agents may be associated with (i.e., covalently or non-covalently) the neutralizing PSGL-1 antibody described herein, or they may be co-administered with or sequentially administered with the antibody. Antibodies linked to a heterologous moiety, such as a polypeptide or an agent, including a second therapeutic agent are also provided, e.g., in U.S. Patent Application Pub. No. 2005/0152906 at paragraphs 123, 124, 148-150, 155, and 157-167, which is incorporated by reference.

The present invention provides for both prophylactic and therapeutic methods of treating a subject, e.g., a human, having or susceptible to a PSGL-1 associated disorder. These disorders may be acute or chronic. For example, stenosis and/or restenosis may be a result of vascular injury, e.g., injury from PTCA, or pathologic injury as it occurs in cardiovascular disease.

In one aspect, the invention provides a method for modulating, e.g., inhibiting, inflammation in a subject by administering to the subject an antibody that specifically binds to PSGL-1. Additionally, methods to modulate thrombosis, coagulation, a T cell response (e.g. a CD8⁺ T cell response), or an immune response are provided. In particular, a PSGL-1 specific antibody optionally modulates binding of PSGL1 to P-selectin, inhibits platelet-leukocyte interaction, microvesicle interaction, microvesicle recruitment, and/or endothelial-leukocyte interaction. Subjects at risk for a PSGL-1 associated disorder include individuals who suffer from cardiovascular disease, individuals with a genetic or epigenetic predisposition, and individuals with an immune disorder, such as a T cell disorder.

Subjects who are at risk also include those who suffer trauma, i.e. accidental, surgical, or non-surgical intervention, such as, e.g. cardiovascular and general vascular procedures or intervention including surgical revascularization, stenting, PCTA or other intervention, which causes vascular injury. Subjects suffering from diabetes mellitus (type 1 diabetes) are at higher risk for restenosis as compared to non-diabetic subjects (see, for example, Van belle et al., Circulation 96:1454-1460(1997); Van Belle et al., Circulation 103:1218-1224 (2001); Stein et al., Circulation 91:979-989 (1995); Levine et al., Am. J. Cardiol. 79:748-755 (1997)). Patients with diabetes mellitus also often have hypercoagulable blood, and intravasal platelet activation may be present in pre-diabetic subjects (Tschoepe et al., Diabetologia 40:573-577 (1997)). Further, a neutralizing antibody may be used to treat multiple sclerosis (MS), a debilitating central nervous system (CNS) disorder. P-selectin and/or PSGL-1 are shown to be critical in the recruitment of leukocytes to the CNS, for example in a model of MS (Picchio et al., J. Immunol. 168:1940-1949(2002); Kerfoot et al., J. Immunol. 169:1000-1006 (2002)).

Elevated levels, e.g., in blood, of endogenous mediators of inflammation are optimally associated with a PSGL-1 associated disorder, such as e.g., an inflammatory disorder. A disorder may be detected and/or assessed by aberrant levels of such endogenous mediators of inflammation or other biomarkers, for example, elevated levels of TNF-α, leukocyte-produced oxidants, procalcitonin, leukocyte high-affinity Fc receptor (CD64), serum C-reactive protein, high mobility group protein 1, plasma D-dimer, IL-1 (e.g., IL-1β), IL-6, IL-8, or platelet activating factor (PAF). For example, a level of TNF-α higher than 25 pg/ml, such as 30, 40, 50, 60, 70, 80, 90, 100, 110, 120, 130, 140, or 150 pg/ml, or a level of C-reactive protein greater than 0.1, 0.2, 0.3, 0.4, 0.5, 0.6, 0.7, 0.8, 0.9, or 1.0 mg/ml may be associated with the disorder. An erythrocyte sedimentation rate (ESR) test, an antinuclear antibody (ANA) test, a rheumatoid factor (RF) test, or a complete blood count (CBD), for example, may also be used to detect PSGL-1 associated disorder, such as, e.g. an inflammatory disorder. Decreased levels of plasminogen, antithrombin III, protein C, thrombomodulin, and endothelial protein C receptor may also be associated with a PSGL-1 associated disorder, such as a thrombotic disorder, for example.

Detection of a reduction in one or more symptoms or clinical manifestations of a PSGL-1 associated disorder, for example, may be used to determine efficacy or disease progression. The antibodies of the present invention can be used to decrease the tendency of the blood to coagulate, which may be useful in the treatment of a PSGL-1 associated disorder. In certain embodiments, the tendency of the blood of an individual to coagulate is reduced at least 10%, such as, e.g., at least 15%, 20%, 30%, 40%, 50%, 60%, 62%, 64%, 66%, 68%, or 70% upon administration of one or more of the presently disclosed antibodies. Suitable assays for measuring blood coagulability will be apparent to one of skill in the art, and include the prothrombin time/international normalized ratio (PT/INR) test, activated partial thromboplastin time (aPTT) test, thrombin time (TT) test, whole blood clotting time test, platelet number and function assays, factor activity assay, reptilase time test, template bleeding time test, activated coagulation time test, and the thromboelastograph (TEG tracing) test. (See e.g., U.S. Patent Pub. No. 2002/0031508 A1, page 26, paragraph 264-275 and page 27, paragraphs 279-281).

Pharmaceutical Compositions

The present invention provides compositions comprising the presently disclosed antibodies. Such compositions may be suitable for pharmaceutical use and administration to patients. The compositions typically comprise one or more antibodies of the present invention and a pharmaceutically acceptable excipient. As used herein, the phrase “pharmaceutically acceptable excipient” includes any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. It may also be possible to obtain compositions which may be topically or orally administered, or which may be capable of transmission across mucous membranes. The administration may, for example, be intravenous, intraperitoneal, intramuscular, intracavity, subcutaneous, or transdermal.

Solutions or suspensions used for intradermal or subcutaneous application typically include one or more of the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes, or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the antibodies can be incorporated with excipients and used in the form of tablets or capsules. Pharmaceutically compatible binding agents, and/or adjuvant materials can be included as part of the composition. The tablets, pills, capsules, and the like can contain any of the following ingredients or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, antibodies are delivered in the form of an aerosol spray from pressured a container or dispenser, which contains a suitable propellant, e.g., a gas such as carbon dioxide, or a nebulizer.

Systemic administration can also be by transmucosal or transdermal means. For example, in case of antibodies that comprise the Fc portion, compositions may be capable of transmission across mucous membranes (e.g., intestine, mouth, or lungs) via the FcRn receptor-mediated pathway (U.S. Pat. No. 6,030,613). Transmucosal administration can be accomplished, for example, through the use of lozenges, nasal sprays, inhalers, or suppositories. For transdermal administration, the active compounds are formulated into ointments, salves, gels, or creams as generally known in the art. For transmucosal or transdermal administration, penetrants appropriate to the barrier to be permeated are used in the formulation. Such penetrants are generally known in the art, and include, for example, detergents, bile salts, and fusidic acid derivatives.

In some instances, oral or parenteral compositions are formulated in dosage unit form for ease of administration and uniformity of dosage. Dosage unit form as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of formulating such an active compound for the treatment of individuals.

Methods to Detect PSGL-1

The antibodies of the present invention may be used to treat, prevent, and/or diagnose a PSGL-1 associated disorder, for example, in a sample from an individual. The primate specific PSGL-1 antibodies may be used to detect the presence of PSGL-1, or fragments thereof, or proteins comprising a primate PSGL-1 epitope, in vivo or in vitro. In some embodiments, the antibodies of the present invention may be used to detect the presence of post-translationally modified (e.g., by sulfation or glycosylation) PSGL-1 or its fragments.

Detection methods are well known in the art and include ELISA, radioimmunoassay, immunoblot, Western blot, immunofluorescence, immunoprecipitation, surface plasmon resonance, and other comparable techniques. The antibodies may further be provided in a diagnostic kit that incorporates one or more of these techniques to detect a peptide or protein comprising PSGL-1. Such a kit may contain other components, packaging, instructions, such as a PSGL-1 protein control, a detection agent, or other material to aid the detection of the protein and/or use of the kit.

Where the antibodies are intended for detection or diagnostic purposes, it may be desirable to modify them, for example, with a ligand group (such as biotin) or a detectable marker group (such as a fluorescent group, a radioisotope or an enzyme). If desired, the antibodies (whether polyclonal or monoclonal) may be labeled using conventional techniques. Suitable labels include fluorophores, chromophores, radioactive atoms, electron-dense reagents, such as heavy metals, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. Other suitable labels may include biotin and avidin or streptavidin, IgG and protein A, and the numerous receptor-ligand couples known in the art. Other permutations and possibilities will be readily apparent to those of ordinary skill in the art, and are considered as equivalents within the scope of the instant invention.

In one embodiment, the PSGL-1 specific antibodies are used in methods to detect PSGL-1, the method comprising adding an antibody that specifically binds PSGL-1 to a biological sample, thereby detecting or quantitating PSGL-1 levels. The presence or absence of PSGL-1 associated disorder may similarly be detected using an antibody described herein. PSGL-1 levels may be used, for example, to detect myeloid leukemia. In a non-linking embodiment, the quantification of surface PSGL-1 differentiates myeloblasts from monoblasts by immunophenotyping (see Kapelmayer et al., Br. J. Haemotol. 115:903-909 (2001)).

In some embodiments, a biological sample is obtained from an individual, and it is optionally prepared and fractionated. Fractionation methods exploit specific cell, tissue, or protein characteristics, such as their inherent chemical properties, including mass, biospecificity, hydrophobicity, charge, or differential location. Protein separation methods include separation by their relative molecular mass in an SDS-PAGE analysis, ion exchange chromatography, size exclusion chromatography, reversed-phase high-performance liquid chromatography ((RP)-HPLC), capillary electrophoresis, capillary isoelectric focusing, and capillary zone electrophoresis, for example. Affinity chromatography is also used to separate or fractionate a biological sample. Separation may be carried out under native or denaturing conditions (see, e.g., Arrell et al., Circulation Res. 88:763-773 (2001)).

Kits to Detect PSGL-1

The invention also provides for a kit for testing a sample for the presence of PSGL-1.

The kit comprises the antibodies of the invention or active portions thereof. The antibody protein can be provided in an appropriate buffer or solvent, or alternatively the antibody protein can be lyophilized, for example. The antibody protein can also be directly or indirectly linked to an agent that aids in visualization of the antibody. For example, the antibody of the invention may be conjugated to a detectable label. The kit optionally comprises a buffer, which can be an aqueous buffer, e.g., PBS. Further, the kit optionally comprises a container, such as a reaction vessel for performing a detection assay. Such a kit may contain other components, packaging, instructions, or other material to aid the detection of the protein.

Screening Methods

Yet another aspect of the invention provides a method of identifying therapeutic agents useful in the treatment of disorders associated with PSGL-1. For example, an agent that modulates increases or decreases) binding of a PSGL-1 specific antibody to its antigen may be identified as a therapeutic agent. Methods to screen for agents useful in treatment of a disorder associated with PSGL-1 are contemplated. Further, methods to screen for agents useful in treating viral infection are contemplated. Appropriate screening assays, e.g., ELISA-based assays, are known in the art. In such a screening assay, a first binding mixture is formed by combining an antibody of the invention and a ligand, e.g., PSGL-1; and the amount of binding between the ligand and the antibody in the first binding mixture (M₀) is measured. A second binding mixture is also formed by combining the antibody, the ligand, and a compound or agent to be screened, and the amount of binding between the ligand and the antibody in the second binding mixture (M₁) is measured. The amounts of binding in the first and second binding mixtures are then compared, for example, by calculating the M₁/M₀ ratio. The compound or agent is considered to be capable of inhibiting binding activity if a decrease in binding in the second binding mixture as compared to the first binding mixture is observed. The formulation and optimization of binding mixtures is within the level of skill in the art; such binding mixtures may also contain buffers and salts necessary to enhance or to optimize binding; and additional control assays may be included in the screening assay of the invention.

Compounds found to reduce the antibody-ligand binding by at least about 10% (i.e., M₁/M₀<0.9), preferably greater than about 20%, 30%, 40%, or 50% may thus be identified and then, if desired, secondarily screened for the capacity to inhibit the activity in other assays, such as the binding to other ligands and other cell-based and in vivo assays as described in the Examples.

The skilled artisan will understand that portions of an immunoglobulin constant region for use in the antibody protein of the invention can include mutants or analogs thereof, or can include chemically modified immunoglobulin constant regions (e.g., pegylation) (see, e.g., Aslam et al., Bioconjugation: Protein Coupling Techniques For the Biomedical Sciences Macmilan Reference, London (1998)) or fragments thereof.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

Isolation of the antibodies of the invention. Single chain Fv fragments (scFv's) were isolated from human phage display libraries using the fully sulfated and glycosylated human PSGL-1 19.ek.Fc fusion protein (SEQ ID NO:42). The PSGL-1 19.ek.Fc construct contains the N-terminal 19 amino acids of human PSGL-1 fused to human immunoglobulin G1 Fc via an enterokinase cleavage site (Somers et al., Cell, 103:467-479 (2000)). A scFv phagemid library, which is an expanded version of the 1.38×10¹⁰ library (Vaughan et al., Nat. Biotechnol. 14:309-314 (1996)), was used to select antibodies that bind to human PSGL-1.

Panning selections were performed as follows. The PSGL-1 19.ek.Fc fusion protein (10 μg/ml in 10 mM NaHCO₃, pH 9.6) or control IgG (50 μg/ml) was coated onto a 96-well plate at 100 μL/well and incubated overnight at 4° C. Wells were washed in PBS and blocked for 1 hour at 37° C. in 3% MPBS (3% ‘Marvel’ skimmed milk powder in PBS). Purified phage (10¹² transducing units) in 100 μL of 3% MPBS also containing 400 μg/ml of the control IgG were added to blocked control IgG wells and incubated at room temperature for 1 hour. The blocked phage were then transferred to the blocked PSGL-1 19.ek.Fc protein coated wells and incubated for 1 hour at room temperature. The wells were first washed 10 times with PBST (PBS containing 0.1% v/v Tween 20), then washed 10 times with PBS. Bound phage particles were eluted with 100 μL of 100 mM triethylamine for 10 minutes at room temperature, then neutralized with 50 μL 1 M Tris HCl, pH 7.4.

The eluted phage particles were used to infect 10 ml of exponentially growing E. coliTG1. The infected cells were grown in 2TY broth for 30 minutes at 37° C. stationary, followed by 30 minutes at 37° C. with aeration. The cells were then streaked onto 2TYAG plates (2TY broth containing 100 μg/ml ampicillin and 2% glucose). The plates were incubated overnight at 30° C. Output colonies were scraped off the plates into 10 ml 2TY broth and 15% glycerol was added for storage at −70° C.

Glycerol stock cultures from the first-round panning selection were super infected with helper phage and rescued to give scFv antibody-expressing phage particles for the second round of panning. Two rounds of panning were carried out in this way.

Soluble selection on PSGL-1 19.ek.Fc was done using biotinylated PSGL-1 19.ek.Fc protein at a concentration of 100 nM. An scFv library, described above, was used. Purified scFv phage (10¹² transducing units) in 1 ml 3% MPBS were blocked for 30 minutes, then biotinylated PSGL-1 19.ek.Fc protein was added, and the sample was incubated at room temperature for 1 hour. Phage/antigen was added to 250 μL of Dynal M280 strepavidin magnetic beads (Dynal, Lake Success, N.Y.) that had been blocked for 1 hour at 37° C. in 1 ml of 3% MPBS, and the sample was incubated an additional 15 minutes at room temperature. The beads were captured using a magnetic rack and washed four times in 1 ml of 3% MPBS/0.1% (v/v) Tween 20, followed by three washes in PBS. After the last PBS wash, the beads were resuspended in 100 μL PBS and used to infect 5 ml of exponentially growing E. coliTG1 cells. Cells and phage were incubated for 1 hour at 37° C. (30 minutes stationary, 30 minutes shaking at 250 rpm), then spread on 2TYAG plates. Plates were incubated at 30° C. overnight and colonies visualized the next day. Output colonies were scraped off the plates and phage rescued as described above.

A second round of soluble selection was then carried out. Output colonies from selections were picked into duplicate 96 well plates containing 1 ml of 2TYAG. Samples were tested either as polyethylene glycol (PEG) precipitated phage supernatants or as crude bacterial periplasmic extracts. Periplasmic scFv production was induced by addition of 1 mM IPTG to exponentially growing cultures and incubation overnight at 30° C. Crude scFv-containing periplasmic extracts were obtained by subjecting the bacterial pellets from the overnight growth to osmotic shock. The pellets were re-suspended in 20% (w/v) sucrose, 1 mM Tris-HCl, pH 7.5 and cooled on ice for 30 minutes. Following centrifugation, the extracts were diluted to 5% in assay buffer (10 mM MOPS, 150 mM NaCl, 1 mM CaCl₂, 1 mM MgCl₂, pH 7.5) and used in the assays.

Phage production was induced by superinfection with helper phage followed by overnight rescue at 30° C. Overnight phage preparations were PEG precipitated before use in the assays. The phage-containing culture supernatants were transferred to a fresh plate and ⅕th volume of 20% (w/v) PEG-8000, 250 mM NaCl was added followed by cooling on ice for 30 minutes. Following centrifugation, the protein pellets were re-suspended in 150 μL assay buffer and were used in the assay at 5%.

ScFv clones that demonstrated the ability to neutralize the binding of biotinylated PSGL-1 19.ek.Fc protein to soluble P-selectin immobilized on plastic in a 96 well plate (ELISA format), were grown in 2TYAG. Periplasmic scFv production was induced by addition of 1 mM isopropylthiogalactoside (IPTG) to exponentially growing cultures at OD₆₀₀=0.9-1.1 and incubated for 3.5 hr at 30° C. Crude scFv-containing periplasmic extracts were obtained by subjecting the bacterial pellets from the 500 mL cultures to osmotic shock. Pellets were resuspended in 20 ml 1 M NaCl, 1 mM EDTA in PBS and cooled on ice for 30 minutes. Following centrifugation, the supernatants containing the scFv were mixed with NiNTA (Qiagen, Valencia, Calif.) and allowed to bind at 4° C. overnight. The NiNTA slurry was loaded onto a polyprep column (Biorad, Cambridge, Mass.), washed, and eluted with PBS containing 250 mM imidazole. The scFv's were concentrated and buffer exchanged to PBS using a Centricon-10 (Millipore, Billerica, Mass.). The scFv protein concentrations were determined using a micro BCA protein assay (Pierce, Rockford, Ill.).

The three scFvs described herein were sequenced using standard DNA sequencing techniques. The nucleic acid and amino acid sequences for PSG3, PSG5, and PSG6 scFv's appear in FIGS. 1, 2, and 3, respectively. Variable domain sequences are indicated in bold, with V_{H} shown in bold and V_{L} shown in bold, underline in parts A and B.

### Example 2

Generation of full-length antibodies. The scFv's were then converted to full length bivalent antibodies (Thompson, J. Immunol. Methods 227:17-29 (1999)). In this context, full-length antibody refers to the single chain antibody reformatted to IgG. The variable heavy and light chains of the selected clones were amplified by PCR from scFv's of Example 1. The PCR primers contained cloning sites which facilitated insertion into the expression vectors. The vector pED6_HC_gamma4 (containing a heavy chain leader sequence and the CH1-CH3 domains of human IgG₄) and the vector pED6_LC (containing a light chain leader sequence and the C domain of human lambda) were transiently expressed in COS cells by TransIt®-based transfection (Mirus Corporation, Madison, Wis.). These vectors are described in Kaufman et al., Nucleic Acids Res. 19:4485-4490 (1991).

For the generation of stable CHO cells, the coding region fragments for the variable heavy and light chains were ligated into separate mammalian expression vectors. CHO 153.8 PA DUKX cells were cotransfected with a lipofectine-based method (Gibco-BRL, Gaithersburg, Md.) after both heavy and light chain plasmids were linearized. Clones were selected and maintained in alpha medium with 10% heat-inactivated, dialyzed fetal calf serum, 2 mM glutamine, 100 U/mL penicillin/streptomycin, and methotrexate ranging from 5 mM to 100 mM.

Clonal CHO lines exhibiting the desired productivity and growth phenotype were selected. The antibody production process was done using chemically defined medium free of animal-derived or human-derived components. The antibodies were purified by Protein A sepharose chromatography (Pharmacia, Uppsala, Sweden), concentrated, and buffer exchanged to PBS pH 7.2 using a Centricon® MW 30 (Millipore, Billerica, Mass.).

### Example 3

Competitive binding assays with PSG3, PSG5, and PSG6. ScFv's and full-length antibodies were screened for the ability to inhibit the binding of biotinylated human PSGL-1 19.ek.Fc fusion protein or biotinylated rPSGL Ig (which contains the N-terminal 47 amino acids of human PSGL-1 fused to human Fc) to P-selectin or L-selectin in competitive enzyme-linked immunosorbent assay (ELISA) format.

Streptavidin-horseradish peroxidase 4 μg/mL (Southern Biotechnology Associates, Birmingham, Ala.) was incubated for 30 minutes at RT with 80 ng/mL biotinylated 19.ek.Fc fusion protein or biotinylated rPSGL-Ig to form a SA-HRP/biotinylated complex (for final concentration of 2 μg/mL SA-HRP, 40 ng/mL biotinylated fusion protein), the complex was then incubated for another 15 minutes at RT in the presence or absence of purified scFv or full length antibodies at different concentrations (for final concentration of 1 μg/mL SA-HRP, 20 ng/mL biotinylated fusion protein).

For these studies, flat microtiter plates (Maxi-Sorp, Nunc, Napeville, Ill.) or Costar (Corning, N.Y.) were coated with human P-selectin-Fc or human L-selectin-Fc at 1 μg/mL, 100 μL per well at 4° C. overnight in coating buffer (10 mM MOPS, 150 mM NaCl, 1 mM CaCl₂, 1 mM MgCl₂, pH 7.5). The next day, plates were washed with coating buffer, 0.05% Tween 20, 50 μg/mL BSA and blocked with 200 μL per well for one hour at RT with coating buffer, 0.1% gelatin (Bio-Rad, Cambridge Mass.). The washed selectin coated plates were incubated for 30 minutes at RT with 100 μL SA-HRP-biotinylated complex with 3 μg/ml scFv's or 1.5 μg/ml mAbs 2× serial diluted. After washing 3 times the wells were incubated 10 minutes with 100 μL TMB (BioFX, Owings Mills, Md.). The reaction was stopped by adding 100 μL 0.18 M H₂SO₄, and the absorbance was read at 450 nm using a plate reader (Lab Systems, Helsinki, Finland).

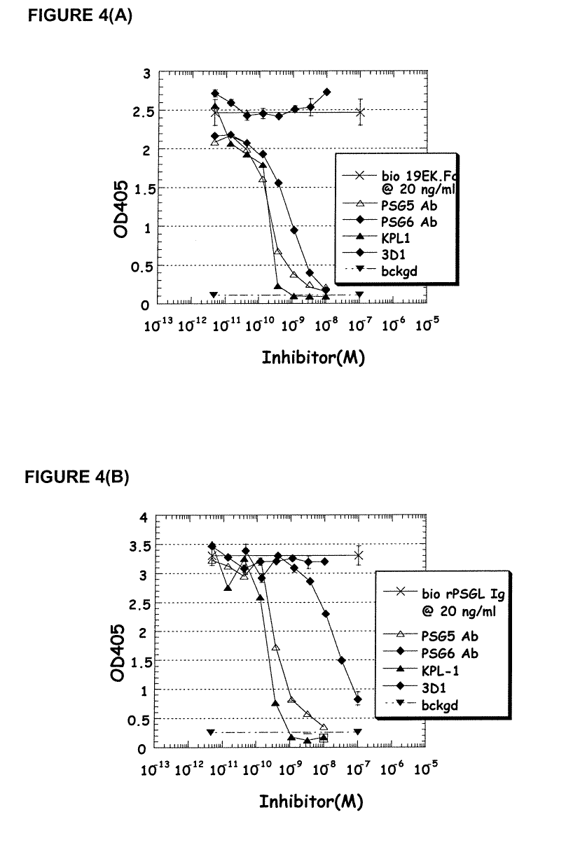

The scFv's showed dose-dependent inhibition of biotinylated PSGL-19.ek.Fc binding to human P-selectin, human L-selectin, and rPSGL-Ig. Thus, the scFv's PSG3, PSG5, and PSG6 competitively inhibited the binding of PSGL-1 to its substrates P-selectin and L-selectin. The binding was specific as shown by lack of an irrelevant antibody 3D1 binding and dose-dependent inhibition of positive control antibody KPL1. The scFvs were converted to intact full-length bivalent antibodies as described in Example 2 (see also, Thompson, J. Immunol. Methods 227:17-29 (1999)). After full-length antibody conversion, the antibodies were tested by competitive ELISA using biotinylated human PSGL-1 19.ek.Fc fusion protein and biotinylated rPSGL Ig (a recombinant PSGL-Ig fusion). The results are shown in FIG. 4(A) (PSGL-19.ek.Fc) and FIG. 4(B) (rPSGL Ig). The specificity of binding was demonstrated by lack of inhibition with the irrelevant 3D1 antibody and a dose-dependent inhibition of positive control antibody KPL1. The binding was also not competitively inhibited by a rat or mouse PSGL-1 sequence, showing the antibodies specifically for human PSGL-1 as compared to rat PSGL 1 (data not shown). The bivalent antibodies demonstrated greater blocking activity relative to their corresponding monovalent scFv forms (data not shown).

### Example 4

Characterization of antibody binding. To elucidate which determinant(s) within the PSGL-1 19.ek.Fc fusion protein were recognized by the human monoclonal antibodies, surface plasmon resonance was performed using a set of highly purified PSGL-1 19.ek peptides with varying degrees of sulfation and/or glycosylation (Somers et al., Cell 103:467-479 (2000)).

The generation of PSGL-1 19.ek peptides has been previously described (Somers et al., Cell 103:467-479 (2000)). Briefly, conditioned media from CHO cells transfected with PSGL-1 19.ek.Fc, Fucosyl transferase VII (FTVII), and CORE-2 cDNAs were purified with Protein A. The purified PSGL-1 19.ek.Fc polypeptide was cleaved by enterokinase treatment. The cleaved protein was separated by Protein A sepharose and the resultant PSGL-1 19.ek peptide pool was resolved by anion exchange HPLC on a SuperQ anion exchange column. (TosoHaas, Montgomeryville, Pa.).

The major PSGL-1 19.ek peptide was the sulfoglycopeptide termed SGP-3, which is posttranslationally modified by sulfate on all three tyrosine residues (i.e., the residues corresponding to Tyr46, Tyr48, and Tyr51of mature human PSGL-1) and by SLe^{x}-capped O-glycan also found in PSGL-1 isolated from HL-60 cells (Wilkins et al;, J. Biol. Chem. 271:18732-42 (1996)). SGP-1 and SGP-2 are forms of hyposulfated forms containing only one and two tyrosine sulfates, respectively. Glycopeptide-1 (GP-1) contains no tyrosine sulfates. Sulfopeptide-1 (SP-1) contains no carbohydrate. These peptides and a synthetic peptide (AnaSpec, San Jose, Calif.) corresponding to the polypeptide portion of SGP-3 but lacking sulfated tyrosine were biotinylated at Lys residues as described previously (Somers et al., Cell 103:467-479, 2000)). The biotinylated peptides were used to characterize the binding of the PSG3, PSG5, and PSG6 antibodies using surface plasmon resonance.

Surface plasmon resonance binding analysis. A BIAcore 2000 instrument (BIAcore AB, Uppsala, Sweden) was used to analyze the interactions between the identified antibodies and biotinylated PSGL-1 19.ek.Fc or derived peptides. Binding experiments were performed at 25° C. using streptavidin-coated sensor chips (BIAcore) and HBS-P buffer (20 mM HEPES [pH 7.4], 150 mM NaCl and 0.005% polysorbate 20 v/v) adjusted to 1 mM for both CaCl₂ and MgCl₂. The streptavidin on the sensor surfaces were condition with three one-minute injections of a solution containing 1 M NaCl and 25 mM NaOH. The chips were regenerated with 5 μL of 0.1% TFA and equilibrated with running buffer. Curves were corrected for non-specific binding by an online baseline subtraction of ligand binding to streptavidin surface in control flow channel. Binding kinetics were analyzed using BIAevaluation software (V2.1; Pharmacia Biosensor, Uppsala, Sweden). The response representing the mass of bound monoclonal antibodies was measured in resonance units (RU). Flow cell one (FC1) was used as the reference surface. The human monoclonal antibodies were diluted in HBS-P buffer at 200 nM and 100 nM based on OD₂₈₀. The diluted antibodies were injected at flow rates of 2, 10, 30, 50, and 100 μL/min to determine the active concentration. Binding kinetics of human anti-PSGL-1 monoclonal antibodies to the immobilized PSGL-1 19.ek.Fc was determined under partial mass transport limitations by triplicate injections at a concentration range (0-100 nM) onto the immobilized biotinlylated PSGL-1 19.ek peptide at a flow rate of 30 μL/min, following injection for two minutes. Dissociation was monitored for ten minutes at the same flow rate. In one experiment, kinetic data for the interaction between monoclonal antibodies and biotinlylated PSGL-1 19.ek.Fc fusion protein found a binding affinity for PSG3 of approximately 1.4×10⁹ M⁻¹, and for PSG6 of approximately 6×10⁸M⁻¹.

Peptide binding. Antibodies (PSG-1, PSG-2, PSG-3, PSG-5, PSG-6, KPL-1, PSL-275, and 3D1) were passed over a streptavidin chip coated with synthetic peptides.

Flow cell 1 (FC1) was left as a blank surface for double reference. The streptavidin chip was coated on flow cell 2 (FC2) with an unglycosylated and unsulfated synthetic peptide 19.ek, that corresponds to the polypeptide portion of SGP-3, and has the amino acid sequence QATEYEYLDYDFLPETEPPRPMMDDDDK (SEQ ID NO:47). The glycopeptide GP-1, or 19.ek having no sulfation was coated on flow cell 3 (FC3). GP-1 has the amino acid sequence QATEYEYLDYDFLPETEPPRPMMDDDDK (SEQ ID NO:47). Sulfated and glycosylated peptide SGP-3 was coated on flow cell 4 (FC4). SGP-3 is the trisulfated glycopeptide 19.ek, and has the amino acid sequence QATEyEyLDyDFLPETEPPRPMMDDDDK (SEQ ID NO:42). Human monoclonal antibodies PSG3, PSG5, and PSG6 as well as PSL-275, KPL1, and an irrelevant human monoclonal 3D1 were injected in duplicate at 100 nM through all flow cells.

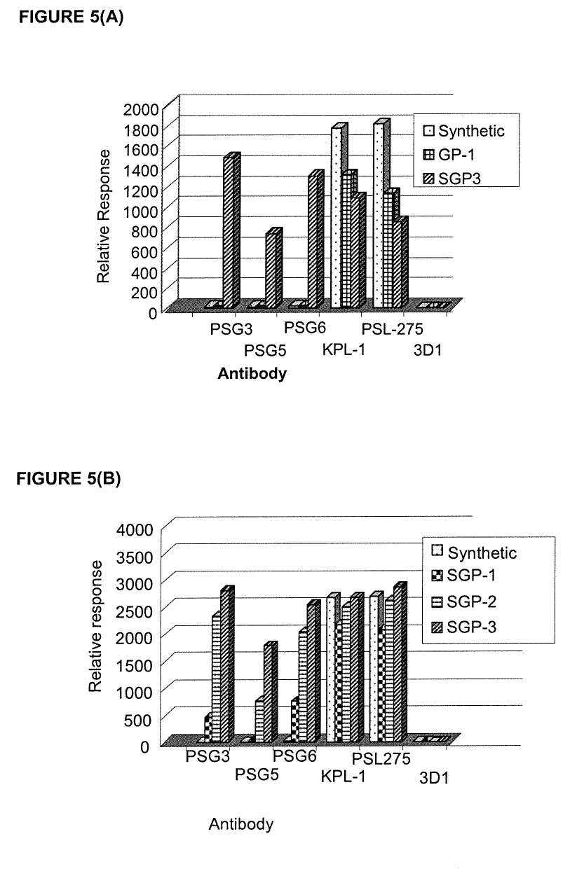

The results are shown in FIG. 5(A). PSL 275 (which is a murine monoclonal anti-human PSGL-1 antibody raised against a human PSGL-1 synthetic peptide) and KPL1 both bound to the synthetic peptide lacking sulfated tyrosine. In contrast, the human monoclonals PSG3, PSG5, and PSG6 did not bind to the synthetic peptide. In addition, the PSG3, PSG5, and PSG6 binding to the glycopeptide, GP-1, was very minimal. The PSG3, PSG5, and PSG6 human monoclonal antibodies required the sulfo-glycopeptide SP-1 in order to bind. These data show that these human monoclonal antibodies recognized PSGL-1 epitope comprising at least one sulfated tyrosine.

To confirm the sulfotyrosine epitope, the antibodies were passed over a streptavidin chip coated with either synthetic peptide, SGP-1, SGP-2, and μSGP-3 using the same conditions for the peptide binding described above.

SGP-1 is the monosulfated glycopeptide 19.ek, and is a mixture of peptides having the amino acid sequences QATEyEYLDYDFLPETEPPRPMMDDDDK (SEQ ID NO:48), QATEYEyLDYDFLPETEPPRPMMDDDK (SEQ ID NO:49), and QATEYEYLDyDFLPETEPPRPMMDDDDK (SEQ ID NO:50). SGP-2 is the disulfated glycopeptide 19.ek, and is a mixture of peptides having the amino acid sequences QATEYEyLDyDFLPETEPPRPMMDDDDK(SEQ ID NO:51), QATEyEYLDyDFLPETEPPRPMMDDDDK (SEQ ID NO:52), and QATEyEyLDYDFLPETEPPRPMMDDDDK (SEQ ID NO:53).

Results are shown in FIG. 5(B). All human monoclonal antibodies required the sulfo-glycopeptide in order to bind. PSG5 binding was significant to SGP-2 and SGP-3. These results confirmed that these human monoclonal antibodies recognized a PSGL-1 epitope comprising the sulfated tyrosines.

### Example 5

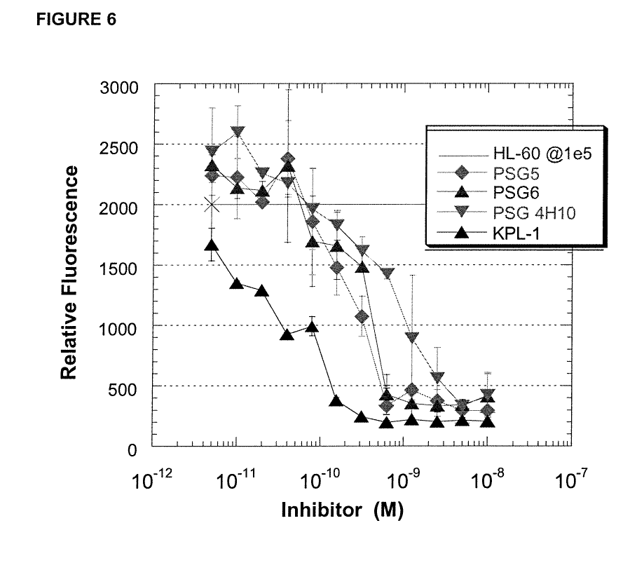

Characterization of the antibodies of the invention using cell adhesion assays. Costar® 3631 plates (Corning Life Sciences, Acton, Mass.) were coated with 100 μL/well of 1.0 μg/mL of soluble human P-selectin (Genetics Institute, Cambridge, Mass.). The plates were incubated at 4° C. (approximately 16 hours). The plates were then blocked for 1 hour at room temperature with 200 μL/well HBSS containing 1.26 mM CaCl₂, 0.64 MgSO₄, 1 mg/ml BSA (Fraction V, SIGMA, St. Louis, Mo.). Just before the addition of the HL-60 cells (ATCC CCL 240), the plates were washed with 200 μL/well HBSS, 0.5 mg/mL BSA. HL-60 cells were labeled with 5 μM calcein-AM (Molecular Probes, Eugene, Oreg.) for 30 minutes at 37° C. Cells were washed and adjusted to a density of 2.0×10⁶ cells/mL in HBSS/1.26 mM CaCl₂, 0.64 MgSO₄. HL-60 cells (100,000/well) were added to the human P-selectin-coated plate in the presence or absence of serial dilutions of PSGL-1 specific antibodies. A baseline reading was performed on the Cytofluor® plate reader (Perspective Biosystems, Framingham, Mass.). The plate was sealed and incubated for 15 minutes at room temperature, and then gently gyrated on a 96-well plate shaker (Lab Line Instruments, Melrose Park, Ill.). The supernatant was removed from the plate and replaced with 100 μL/well of HBSS/2 mM CaCl₂. Washing by gentle gyration and buffer replacement was repeated four to six times with intervening plate readings. For each well, the percentage of adherent cells was calculated as the percentage of cell binding as compared to the baseline readings. The mean and standard deviation were calculated from all multiple wells for each condition. PSG3, PSG5, and PSG6 showed dose-dependent inhibition of HL-60 binding to human P-selectin (See FIG. 6).

### Example 6

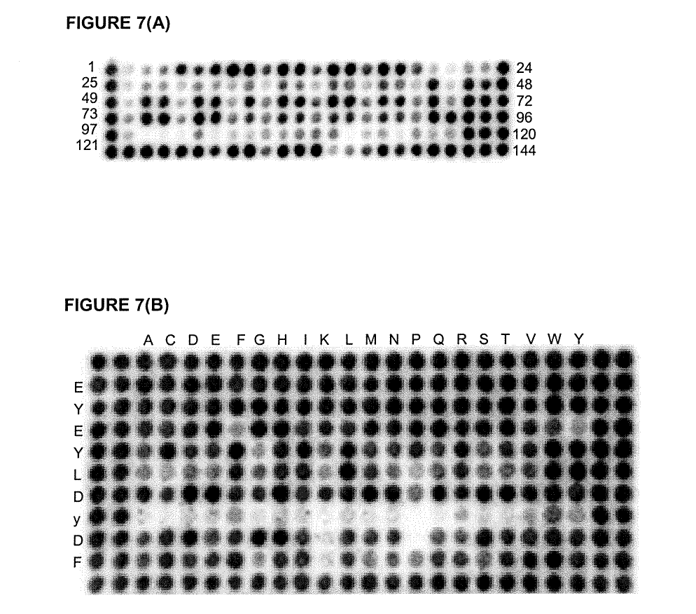

Characterization of the antibodies using FACS analysis. The binding specificity of the neutralizing antibodies was assessed by FACS analysis of HL-60 cells as well as in parent and recombinant CHO cells expressing human or rat PSGL-1 (data not shown). For FACS analysis, 0.5×10⁶ cells were incubated in 100 μL HBSS 2 mM CaCl₂, 1% fetal calf serum, NaN₃, containing 15 or 50 μg of the indicated scFvs complexed with 50 μg/ml mouse-anti-Myc-HRP (Roche Applied Sciences, Indianapolis, Ind.). The cells were washed and incubated with 1:100 anti-mouse FITC-conjugated secondary antibody (Roche Applied Sciences, Indianapolis, Ind.). Analysis was performed on viable cells of the mononuclear population with low forward and side scatter properties. As shown in FIG. 7(A) and FIG. 7(B), PSG3, PSG5, and PSG6 scFvs bind to native PSGL-1 expressed on HL-60 cells. In CHO cells, the full length PSG3 and PSG6 antibodies did not bind the parent CHO cell line or recombinant CHO cells expressing rat PSGL-1, but bound to the recombinant CHO cells expressing human PSGL-1. (In this context, full-length PSG3 refers to the single chain antibody reformatted to IgG.) PSG5 showed some binding to the parent CHO line and the recombinant cells expressing rat PSGL-1, and showed the strongest binding to recombinant cells expressing human PSGL-1 (data not shown).

### Example 7

Epitope mapping of PSG3. Fmoc-protected amino acids and cellulose membranes modified with polyethylene glycol were purchased from Intavis. Fmoc-protected β-alanine was purchased from Chem-Impex (Wood Dale, Ill.). The arrays were defined on the membranes by coupling a β-alanine spacer, followed by elongation of the peptide chain. Peptides were synthesized using standard DIC/HOBt coupling chemistry as described previously. (See, e.g., Molina et al., Pept. Res. 9:151-155 (1996) and Frank et al., Tetrahedron 48:9217-9232 (1992)). Activated amino acids were spotted using an Abimed ASP 222 robot. Washing and deprotection steps were done manually and the peptides were N-terminally acetylated after the final synthesis cycle.

Following peptide synthesis and side chain deprotection, the membranes were washed in methanol for 10 minutes and in blocker (1% casein in TBS) for 10 minutes. The membranes were then incubated with 1 μg/mL of PSG2 in TBS for 1 hour with gentle shaking. The membranes were washed 4 times for 2 minutes in TBS and then probed with an HRP-conjugated anti-Fc antibody in blocker. After washing with TBS, bound protein was visualized using SuperSignal West reagent (Pierce) and a digital camera (AlphaInnotech FluorImager). Signal intensity reflects the amount of protein bound at each spot.

The binding epitope for PSG3 was mapped using the peptides of Table 4.

TABLE 4

FIG. 7(A)

| | | |
|---|---|---|
| Peptide No. | Peptide Sequence | SEQ ID NO. |

| | | |
|---|---|---|
| 1 | QATEyEyLDyDFL | 54 |
| 2 | QATEYEYLDYDFL | 55 |
| 3 | QATEyEYLDYDFL | 56 |
| 4 | QATEYEyLDYDFL | 57 |
| 5 | QATEYEYLDyDFL | 58 |
| 6 | QATEyEyLDYDFL | 59 |
| 7 | QATEyEYLDyDFL | 60 |
| 8 | QATEYEyLDyDFL | 61 |
| 9 | QATEyEyLDyDF | 62 |
| 10 | QATEyEyLDyD | 63 |
| 11 | ATEyEyLDyDFL | 64 |
| 12 | ATEyEyLDyDF | 65 |
| 13 | ATEyEyLDyD | 66 |
| 14 | TEyEyLDyDFL | 67 |
| 15 | TEyEyLDyDF | 68 |
| 16 | TEyEyLDyD | 69 |
| 17 | EyEyLDyDFL | 70 |
| 18 | EyEyLDyDF | 71 |
| 19 | EyEyLDyD | 72 |
| 20 | QATEyEYLDYDF | 73 |
| 21 | QATEyEYLDYD | 74 |
| 22 | ATEyEYLDYDFL | 75 |
| 23 | ATEyEYLDYDF | 76 |
| 24 | QATEyEyLDyDFL | 77 |
| 25 | QATEyEyLDyDFL | 78 |
| 26 | ATEyEYLDYD | 79 |
| 27 | TEyEYLDYDFL | 80 |
| 28 | TEyEYLDYDF | 81 |
| 29 | TEyEYLDYD | 82 |
| 30 | EyEYLDYDFL | 83 |
| 31 | EyEYLDYDF | 84 |
| 32 | EyEYLDYD | 85 |
| 33 | QATEYEyLDYDF | 86 |
| 34 | QATEYEyLDYD | 87 |
| 35 | ATEYEyLDYDFL | 88 |
| 36 | ATEYEyLDYDF | 89 |
| 37 | ATEYEyLDYD | 90 |
| 38 | TEYEyLDYDFL | 91 |
| 39 | TEYEyLDYDF | 92 |
| 40 | TEYEyLDYD | 93 |
| 41 | EYEyLDYDFL | 94 |
| 42 | EYEyLDYDF | 95 |
| 43 | EYEyLDYD | 96 |
| 44 | QATEYEYLDyDF | 97 |
| 45 | QATEYEYLDyD | 98 |
| 46 | ATEYEYLDyDFL | 99 |
| 47 | ATEYEYLDyDF | 100 |
| 48 | QATEyEyLDyDFL | 101 |
| 49 | QATEyEyLDyDFL | 102 |
| 50 | ATEYEYLDyD | 103 |
| 51 | TEYEYLDyDFL | 104 |
| 52 | TEYEYLDyDF | 105 |
| 53 | TEYEYLDyD | 106 |
| 54 | EYEYLDyDFL | 107 |
| 55 | EYEYLDyDF | 108 |
| 56 | EYEYLDyD | 109 |
| 57 | QATEyEYLDyDF | 110 |
| 58 | QATEyEYLDyD | 111 |
| 59 | ATEyEYLDyDFL | 112 |
| 60 | ATEyEYLDyDF | 113 |
| 61 | ATEyEYLDyD | 114 |
| 62 | TEyEYLDyDFL | 115 |
| 63 | TEyEYLDyDF | 116 |
| 64 | TEyEYLDyD | 117 |
| 65 | EyEYLDyDFL | 118 |
| 66 | EyEYLDyDF | 119 |
| 67 | EyEYLDyD | 120 |
| 68 | QATEYEyLDyDF | 121 |
| 69 | QATEYEyLDyD | 122 |
| 70 | ATEYEyLDyDFL | 123 |
| 71 | ATEYEyLDyDF | 124 |
| 72 | QATEyEyLDyDFL | 125 |
| 73 | QATEyEyLDyDFL | 126 |
| 74 | ATEYEyLDyD | 127 |
| 75 | TEYEyLDyDFL | 128 |
| 76 | TEYEyLDyDF | 129 |
| 77 | TEYEyLDyD | 130 |
| 78 | EYEyLDyDFL | 131 |
| 79 | EYEyLDyDF | 132 |
| 80 | EYEyLDyD | 133 |
| 81 | QATEyEyLDYDF | 134 |
| 82 | QATEyEyLDYD | 135 |
| 83 | ATEyEyLDYDFL | 136 |
| 84 | ATEyEyLDYDF | 137 |
| 85 | ATEyEyLDYD | 138 |
| 86 | TEyEyLDYDFL | 139 |
| 87 | TEyEyLDYDF | 140 |
| 88 | TEyEyLDYD | 141 |
| 89 | EyEyLDYDFL | 142 |
| 90 | EyEyLDYDF | 143 |
| 91 | EyEyLDYD | 144 |
| 92 | QATEyEyLDyDFL | 145 |
| 93 | QATEyEyLDyDFL | 146 |
| 94 | QATEyEyLDyDFL | 147 |
| 95 | QATEyEyLDyDFL | 148 |
| 96 | QATEyEyLDyDFL | 149 |
| 97 | QATEyEyLDyDFL | 150 |
| 98 | DDFEDPDyTyNTD | 151 |
| 99 | DDFEDPDYTYNTD | 152 |
| 100 | DDFEDPDyTYNTD | 153 |
| 101 | DDFEDPDYTyNTD | 154 |
| 102 | DFEyPDySVyGTD | 155 |
| 103 | DFEYPDYSVYGTD | 156 |
| 104 | DFEyPDYSVYGTD | 157 |
| 105 | DFEYPDySVYGTD | 158 |
| 106 | DFEYPDYSVyGTD | 159 |
| 107 | DFEyPDySVYGTD | 160 |
| 108 | DFEyPDYSVyGTD | 161 |
| 109 | DFEYPDySVyGTD | 162 |
| 110 | GDTDLyDyyPEED | 163 |
| 111 | GDTDLYDYYPEED | 164 |
| 112 | GDTDLyDYYPEED | 165 |
| 113 | GDTDLYDyYPEED | 166 |
| 114 | GDTDLYDYyPEED | 167 |
| 115 | GDTDLyDyYPEED | 168 |
| 116 | GDTDLyDYyPEED | 169 |
| 117 | GDTDLYDyyPEED | 170 |
| 118 | QATEyEyLDyDFL | 171 |
| 119 | QATEyEyLDyDFL | 172 |
| 120 | QATEyEyLDyDFL | 173 |
| 121 | QATEyEyLDyDFL | 174 |
| 122 | AATEyEyLDyDFL | 175 |
| 123 | QAAEyEyLDyDFL | 176 |
| 124 | QATAyEyLDyDFL | 177 |
| 125 | QATEAEyLDyDFL | 178 |
| 126 | QATEyAyLDyDFL | 179 |
| 127 | QATEyEALDyDFL | 180 |
| 128 | QATEyEyADyDFL | 181 |
| 129 | QATEyEyLAyDFL | 182 |
| 130 | QATEyEyLDADFL | 183 |
| 131 | QATEyEyLDyAFL | 184 |
| 132 | QATEyEyLDyDAL | 185 |
| 133 | QATEyEyLDyDFA | 186 |
| 134 | QATEYEYLDYDFL | 187 |
| 135 | QATEyEYLDYDFL | 188 |
| 136 | QATEYEyLDYDFL | 189 |
| 137 | QATEYEYLDyDFL | 190 |
| 138 | QATEyEyLDYDFL | 191 |
| 139 | QATEyEYLDyDFL | 192 |
| 140 | QATEYEyLDyDFL | 193 |
| 141 | QATEyEyLDyDFL | 194 |
| 142 | QATEyEyLDyDFL | 195 |
| 143 | QATEyEyLDyDFL | 196 |
| 144 | QATEyEyLDyDFL | 197 |

As demonstrated in FIGS. 7(A) and 7(B), epitope mapping analysis indicates that sulfated tyrosine is an essential part of the epitope recognized by the PSG3 antibody. High affinity binding of the PSG3 antibody can occur with the peptide sequence EYEYLDyDF (SEQ ID NO:45). A comparison of the binding of PSG3 to peptide 31 vs. peptide 55 in FIG. 7(A) indicates that PSG3 binds poorly to the peptide sequence EyEYLDYDF (SEQ ID NO:44). This demonstrates that when this peptide sequence contains a single sulfated tyrosine, the position of the sulfated tyrosine in the sequence is critical for optimal binding.

### Example 8

Inhibition of PSGL-1 procoagulant activity. Whole blood samples from five donors were incubated individually for 6 hours at 37° C. with or without human P-selectin-Ig. Fibrin formation in the plasma of samples treated with P-selectin-Ig was significantly faster than controls, resulting in faster whole blood and plasma clotting times. Addition of an inhibitory antibody to human PSGL-1 (PSG3-G1) completely inhibited the observed increases in whole blood and plasma clotting time. These results demonstrate that inhibiting PSGL-1 interaction with P-selectin can block the procoagulant activity of P-selectin in vitro and suggest that similar approaches may have therapeutic value in vivo (Hrachovinova et al., Nature Med. 9:1020-1025 (2003)).

### Example 9

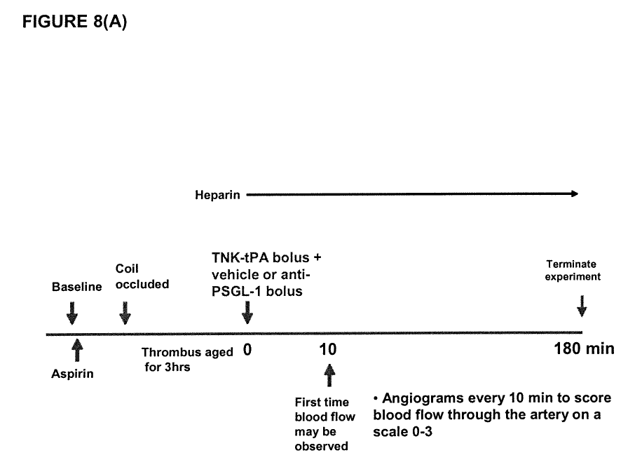

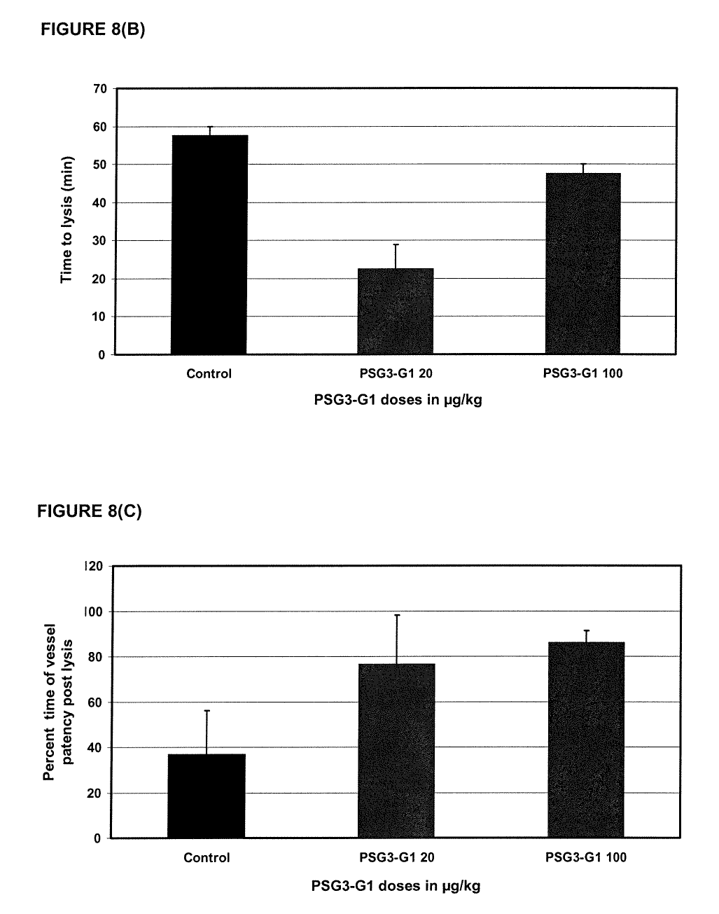

Acceleration of thrombolysis in non-human primates. As depicted in FIG. 8(A), primates (Cynomolgus monkeys, n=3 per treatment group) were given an intravenous dose of aspirin at 5 mg/kg. A thrombus was induced by copper coil placement under fluoroscopic guidance in the femoral artery of a non-human primate and monitored by angiography. Heparin infusion of 70 U/kg heparin was initiated and continued throughout the experiment and was adjusted to keep activated clotting time (measured every 15 minutes) at 2-fold to 3-fold of baseline. A formed thrombus was allowed to age for 180 minutes and an angiogram was taken to confirm vascular occlusion. TNKase (Tenecteplase, Genentech) was then dosed via single bolus at 0.5 mg/kg and in combination with either control saline or 20 μg/kg PSG3-G1 antibody. Blood flow was monitored via angiography at 10 minute intervals. As can be seen in FIG. 8(B), as compared to the control group, the animals dosed with a PSG3 antibody demonstrated accelerated time to lysis. Treatment increased the average time to lysis by 36 minutes. FIG. 8(C) shows that the PSG3 treated animals recovered vessel patency (TIMI grade flow of 2/3) more quickly after lysis of aged thrombus than controls.

### Example 10

Treatment of stenosis or restenosis in humans. An individual having or susceptible to stenosis or restenosis (e.g., resulting from a vascular or pathologic injury) is treated with at least one PSGL-1 specific antibody such as PSG3, PSG5, or PSG6. The PSGL-1 specific antibody is administered intravenously or by injection in an efficacious quantity at dosages chosen from 1 μg/kg to 10 μg/kg, 10 μg/kg to 100 μg/kg, 100 μg/kg to 1 mg/kg, 1mg/kg to 10 mg/kg, and 10 mg/kg to 30 mg/kg body weight. Administration of the anti-PSGL-1 antibody results in improvement of one or more clinical manifestations of stenosis or restenosis.

### Example 11

Treatment of sepsis in humans. An individual having sepsis (e.g., sepsis resulting from a bacterial, viral, fungal, or parasitic infection) is treated with at least one neutralizing PSGL-1 specific antibody such as PSG3, PSG5, or PSG6. The sulfotyrosine specific antibody is administered intravenously or by any other suitable method, and may be administered in an efficacious quantity between 1 μg/kg to 10μg/kg, 10 μg/kg to 100 μg/kg, 100 μg/kg to 1 mg/kg, 1 mg/kg to 10 mg/kg, and 10 mg/kg to 30 mg/kg body weight. The antibody is optionally administered in combination with one or more antibiotic, antiviral, antifungal, antiparasitic, anti-inflammatory, or blood pressure raising agents. Administration of the PSGL-1 specific antibody results in a decrease in blood coagulability and reduction of at least one of the symptoms or clinical indicators of sepsis.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. Each numerical parameter should also be construed in light of the number of significant digits and ordinary rounding approaches.

Modifications and variations of this invention can be made without departing from its spirit and scope, as will be apparent to those skilled in the art. The specific embodiments described herein are exemplary, and are not meant to be limiting in any way.

1. An isolated antibody comprising at least one amino acid sequence chosen from SEQ ID NOs:2, 4, 6, 8, 10, 12, 14, 16, 18, 19, 20, 21, 22, 23, 24, 25, 26, 27, 28, 29, 30, 31, 32, 33, 34, 35, 36, 37, 205, 206, 207, and 208 wherein the antibody specifically binds to primate PSGL-1 and reduces a PSGL-1 activity.

2. The antibody of claim 1, wherein the antibody specifically binds to a primate PSGL-1 comprising SEQ ID NO:42.

3. The antibody of claim 1, wherein the antibody specifically binds with an affinity constant greater than 10⁸ M⁻¹.

4. The antibody of claim 1, wherein the antibody is monoclonal.

5. The antibody of claim 1, wherein the antibody is human.

6. The antibody of claim 1, wherein the antibody specifically binds to primate PSGL-1 comprising a sulfotyrosine, but does not specifically bind to the corresponding PSGL-1 comprising an unmodified tyrosine.

7. An isolated antibody of claim 1, that specifically binds to SEQ ID NO:42, but not SEQ ID NO:47.

8. A pharmaceutical composition comprising the antibody of claim 1.

9. An isolated nucleic acid encoding the antibody of claim 1.

10. An isolated nucleic acid chosen from at least one nucleic acid comprising:
(a) SEQ ID NO:1, 3 or 5;
(b) a nucleic acid that encodes SEQ ID NOs:2, 4, 6, 8, 10, 12, 14, 16, 18, 19, 20, 21, 22, 23, 24, 25, 26, 27, 28, 29, 30, 31, 32, 33, 34, 35, or 36; and
(c) a nucleic acid capable of hybridization to the complement of a nucleic acid of (a) or (b) under conditions of high stringency and which encodes an antibody that specifically binds to primate PSGL-1.

11. An expression vector comprising the nucleic acid of claim 10.

12. A host cell comprising the vector of claim 11.

13. A method of making an antibody that specifically binds to primate PSGL-1 comprising:
(a) transforming a cell with a DNA construct comprising a nucleic acid of claim 10;
(b) culturing the transformed cell under conditions where an antibody is expressed; and
(c) isolating the antibody that specifically binds to primate PSGL-1.

14. The method of claim 13, wherein the antibody is a monovalent antibody.

15. The method of claim 13, wherein the antibody is a bivalent antibody.

16. A method to produce an antibody that specifically binds to primate PSGL-1 comprising:
(a) providing a repertoire of nucleic acids encoding a variable domain that either includes a CDR3 to be replaced or lacks a CDR3 encoding region;
(b) combining the repertoire with a donor nucleic acid encoding an amino acid sequence substantially identical to SEQ ID NO:21, 27, or 33 such that the donor nucleic acid is inserted into the CDR3 region in the repertoire, so as to provide a product repertoire of nucleic acids encoding a variable domain;
(c) expressing the nucleic acids of said product repertoire; and
(d) selecting an antigen-binding fragment that specifically binds to PSGL-1.

17. A method to identify an agent that modulates primate PSGL-1 comprising:
(a) combining the antibody of claim 1 with a ligand, wherein the ligand comprises a PSGL-1 protein or a fragment thereof that specifically binds to the antibody;
(b) detecting modulation of the binding between the ligand and the antibody in the presence and absence of the agent; and
(c) thereby identifying an agent that modulates the PSGL-1 protein.

18. A method to detect a primate PSGL-1 in a biological sample, comprising
(a) adding an antibody of claim 1 to a biological sample;
(b) adding a detectable label; and
(c) detecting the amount of the antibody that specifically binds to the sample.

19. A diagnostic method to detect primate PSGL-1 or sulfated PSGL-1 peptides in a biological sample, comprising contacting a biological sample with an antibody of claim 1.

20. A method to quantify the amount of primate PSGL-1 in a biological sample, comprising adding an antibody of claim 1 to a biological sample.

21. A kit for detecting primate PSGL-1 comprising the antibody of claim 1.

22. A method for treating a PSGL-1 associated disorder, comprising administering to an individual an effective dose of the antibody of claim 1.

23. The method of claim 22, wherein the PSGL-1 associated disorder is a disorder associated with inflammation.

24. The method of claim 22, wherein the PSGL-1 associated disorder is a disorder associated with thrombosis.

25. The method of claim 22, wherein the PSGL-1 associated disorder is a disorder associated with coagulation.

26. The method of claim 25, wherein the disorder is associated with a T cell response.

27. The method of claim 22, wherein the PSGL-1 associated disorder is a cardiovascular disorder.

28. The method of claim 22, wherein the individual is a primate.

29. The method of claim 23, wherein the mammal is a human.
